(19) United States
(12) Patent Application Publication
(10) Pub. No.: US 2011/0184026 A1
(43) Pub. Date: Jul. 28, 2011

(54) C4-SUBSTITUTED ALPHA-KETO OXAZOLES

(76) Inventor: Dale L. Boger, La Jolla, CA (US)

(21) Appl. No.: 12/999,442

(22) PCT Filed: Jun. 19, 2009
(86) PCT No.: PCT/US09/03680

§ 371 Date: Apr. 15, 2011

Related U.S. Application Data
(60) Provisional application No. 61/074,086, filed on Jun. 19, 2008.

(51) Int. Cl. A61K 31/4439; C07D 263/34; A61K 31/421; C07D 263/46; C07D 413/04; A61P 29/00; A61P 25/00; A61P 1/08; A61P 19/02; A61P 1/00; A61P 3/10; A61P 11/00; A61P 15/10; A61P 31/14; C12N 9/99

(52) U.S. Cl. 514/340; 548/236; 514/374; 548/228; 514/376; 546/271.4; 435/184

(57) ABSTRACT

The invention provides a series of C4-substituted oxazole compounds having an alpha keto side chain at the 2 position, for example, compounds of formula I. The compounds can inhibit fatty acid amide hydrolase and can be useful for treatment of malconditions modulated by fatty acid amide hydrolase. The invention further provides methods of making compounds of formula I, useful intermediates in the preparation of compounds of formula I, and methods of using compounds of formula I and compositions thereof.

### CLAIM OF PRIORITY

This application claims priority to U.S. Provisional Application No. 61/074,086, filed on Jun. 19, 2008, which is incorporated herein by reference.

### GOVERNMENT SUPPORT

This invention was made with government support under Grant No. DA15648 awarded by the National Institutes of Health. The United States Government has certain rights in the invention.

### BACKGROUND OF THE INVENTION

Medicinal benefits have been attributed to the cannabis plant for centuries. The primary bioactive constituent of cannabis is Δ⁹-tetrahydrocannabinol (THC). The discovery of THC eventually led to the identification of two endogenous cannabinoid receptors responsible for its pharmacological actions, namely CB₁ and CB₂ (Goya, Exp. Opin. Ther. Patents 2000, 10, 1529). These discoveries not only established the site of action of THC, but also inspired inquiries into the endogenous agonists of these receptors, or “endocannabinoids”. The first endocannabinoid identified was the fatty acid amide anandamide (AEA). AEA itself elicits many of the pharmacological effects of exogenous cannabinoids (Piomelli, Nat. Rev. Neurosci. 2003, 4(11), 873).

The catabolism of AEA is primarily attributable to the integral membrane bound protein fatty acid amide hydrolase (FAAH), which hydrolyzes AEA to arachidonic acid. FAAH was characterized in 1996 by Cravatt and co-workers (Cravatt, Nature 1996, 384, 83). It was subsequently determined that FAAH is additionally responsible for the catabolism of a large number of important lipid signaling fatty acid amides including: another major endocannabinoid, 2-arachidonoylglycerol (2-AG) (Science 1992, 258, 1946-1949); the sleep-inducing substance, oleamide (OEA) (Science 1995, 268, 1506); the appetite-suppressing agent, N-oleoylethanolamine (Rodriguez de Fonesca, Nature 2001, 414, 209); and the anti-inflammatory agent, palmitoylethanolamide (PEA) (Lambert, Curr. Med. Chem. 2002, 9(6), 663).

Small-molecule inhibitors of FAAH should elevate the concentrations of these endogenous signaling lipids and thereby produce their associated beneficial pharmacological effects. There have been some reports of the effects of various FAAH inhibitors in pre-clinical models.

In particular, two carbamate-based inhibitors of FAAH were reported to have analgesic properties in animal models. In rats, BMS-1 (see WO 02/087569), was reported to have an analgesic effect in the Chung spinal nerve ligation model of neuropathic pain, and the Hargraves test of acute thermal nociception. URB-597 was reported to have efficacy in the zero plus maze model of anxiety in rats, as well as analgesic efficacy in the rat hot plate and formalin tests (Kathuria, Nat. Med. 2003, 9(1), 76). The sulfonylfluoride AM374 was also shown to significantly reduce spasticity in chronic relapsing experimental autoimmune encephalomyelitis (CREAE) mice, an animal model of multiple sclerosis (Baker, FASEB J. 2001, 15(2), 300).

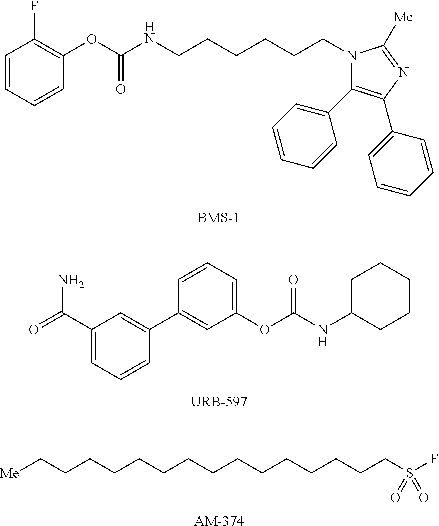

In addition, the oxazolopyridine ketone OL-135 is reported to be a potent inhibitor of FAAH, and has been reported to have analgesic activity in both the hot plate and tail emersion tests of thermal nociception in rats (WO 04/033652).

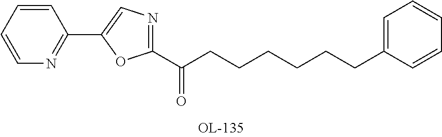

Results of research on the effects of certain exogenous cannabinoids has elucidated that an FAAH inhibitor may be useful for treating various conditions, diseases, disorders, or symptoms. These include pain, nausea/emesis, anorexia, spasticity, movement disorders, epilepsy and glaucoma. To date, approved therapeutic uses for cannabinoids include the relief of chemotherapy-induced nausea and emesis among patients with cancer and appetite enhancement in patients with HIV/AIDS who experience anorexia as a result of wasting syndrome. Two products are commercially available in some countries for these indications, namely, dronabinol (Marinol®) and nabilone.

Apart from the approved indications, a therapeutic field that has received much attention for cannabinoid use is analgesia, i.e., the treatment of pain. Five small randomized controlled trials showed that THC is superior to placebo, producing dose-related analgesia (Robson, Br. J. Psychiatry 2001, 178, 107-115). Atlantic Pharmaceuticals is reported to be developing a synthetic cannabinoid, CT-3, a 1,1-dimethyl heptyl derivative of the carboxylic metabolite of tetrahydrocannabinol, as an orally active analgesic and anti-inflammatory agent. A pilot phase II trial in chronic neuropathic pain with CT-3 was reported to have been initiated in Germany in May 2002.

A number of individuals with multiple sclerosis have claimed a benefit from cannabis for both disease-related pain and spasticity, with support from small controlled trials (Svendsen, Br. Med. J. 2004, 329, 253). Likewise, various victims of spinal cord injuries, such as paraplegia, have reported that their painful spasms are alleviated after smoking marijuana. A report showing that cannabinoids appear to control spasticity and tremor in the CREAE model of multiple sclerosis demonstrated that these effects are mediated by CB₁ and CB₂ receptors (Baker, Nature 2000, 404, 84-87). Phase 3 clinical trials have been undertaken in multiple sclerosis and spinal cord injury patients with a narrow ratio mixture of tetrahydrocannabinol/cannabidiol (THC/CBD).

Reports of small-scale controlled trials have been conducted to investigate other potential commercial uses of cannabinoids have been made. Trials in volunteers have been reported to have confirmed that oral, injected and smoked cannabinoids produced dose-related reductions in intraocular pressure (IOP) and therefore may relieve glaucoma symptoms. Ophthalmologists have prescribed cannabis for patients with glaucoma in whom other drugs have failed to adequately control intraocular pressure (Robson, 2001).

Inhibition of FAAH using a small-molecule inhibitor may be advantageous compared to treatment with a direct-acting CB₁ agonist. Administration of exogenous CB₁ agonists may produce a range of responses, including reduced nociception, catalepsy, hypothermia, and increased feeding behavior. These four in particular are termed the “cannabinoid tetrad.” Experiments with FAAH−/−mice show reduced responses in tests of nociception, but did not show catalepsy, hypothermia, or increased feeding behavior (Cravatt, Proc. Natl. Acad. Sci. USA 2001, 98(16), 9371). Fasting caused levels of AEA to increase in rat limbic forebrain, but not in other brain areas, providing evidence that stimulation of AEA biosynthesis may be anatomically regionalized to targeted CNS pathways (Kirkham, Br. J. Pharmacol. 2002, 136, 550). The finding that AEA increases are localized within the brain, rather than systemic, suggests that FAAH inhibition with a small molecule could enhance the actions of AEA and other fatty acid amides in tissue regions where synthesis and release of these signaling molecules is occurring in a given pathophysiological condition (Piomelli, 2003).

In addition to the effects of a FAAH inhibitor on AEA and other endo-cannabinoids, inhibitors of FAAH's catabolism of other lipid mediators may be used in treating other therapeutic indications. For example, PEA has demonstrated biological effects in animal models of inflammation (Holt, et al. Br. J. Pharmacol. 2005, 146, 467-476), immunosuppression, analgesia, and neuroprotection (Ueda, J. Biol. Chem. 2001, 276(38), 35552). Oleamide, another substrate of FAAH, induces sleep (Boger, Proc. Natl. Acad. Sci. USA 2000, 97(10), 5044; Mendelson, Neuropsychopharmacology 2001, 25, S36). Inhibition of FAAH has also been implicated in cognition (Varvel, et al. J. Pharmacol. Exp. Ther. 2006, 317(1), 251-257) and depression (Gobbi, et al. Proc. Natl. Acad. Sci. USA 2005, 102(51), 18620-18625).

Thus, there is evidence that small-molecule FAAH inhibitors may be useful in treating pain of various etiologies, anxiety, multiple sclerosis and other movement disorders, nausea/emesis, eating disorders, epilepsy, glaucoma, inflammation, immunosuppression, neuroprotection, depression, cognition enhancement, and sleep disorders, and potentially with fewer side effects than treatment with an exogenous cannabinoid.

Various small-molecule FAAH modulators have been described, e.g., in U.S. Patent Application Publication No. US 2006/0100212, U.S. patent application Ser. No. 11/708,788 (filed Feb. 20, 2007), and U.S. Provisional Patent Appl. No. 60/843,277 (filed Sep. 8, 2006). However, there remains a need for potent and/or selective FAAH modulators with suitable pharmaceutical properties.

### SUMMARY

A series of C4 substituted α-ketooxazoles were discovered that inhibit the serine hydrolase fatty acid amide hydrolase and provide FAAH-modulating activity. Accordingly, the invention provides a compound of formula I:

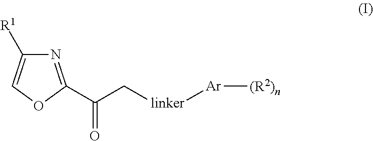

wherein

R¹ is —Y—R^{x};

Y is —CH₂—, —O—, —CH₂O—, —OCH₂—, —C(═O)—, —CO₂—, —OC(═O)—, —S—, —S(O)—, —S(O)₂—, —N(R^{a})—, or a direct bond;

R^{x} is H, halo, (C₁-C₂₀)alkyl, (C₁-C₈)cycloalkyl, trifluoromethyl, aryl, heteroaryl, —CN, —NO₂, or —NR^{a}R^{b};

linker is a (C₁-C₂₀)alkyl chain wherein one to five carbons of the chain are optionally be replaced with O or S, or linker is a direct bond;

Ar is (C₆-C₁₄)aryl;

each R² is independently H, —X—R³, or —X-Ph-X—R³;

n is 1-4;

each X is independently —CH₂—, —O—, —CH₂O—, —OCH₂—, —C(═O)—, —CO₂—, —OC(═O)—, —S—, —S(O)—, —S(O)₂—, —N(R^{a})—, or a direct bond;

each R³ is independently H, (C₁-C₈)alkyl, (C₃-C₈)cycloalkyl, aryl, heteroaryl, —CF₃, —CN, —C(O)(C₁-C₈)alkyl optionally substituted with one, two, or three fluoro substituents, —CO₂(C₁-C₈)alkyl, —CO₂H, —C(O)NR^{a}R^{b}, —OH, —O(C₁-C₈)alkyl, -halo, —NO₂, —NR^{a}R^{b}, —N(R^{a})C(O)R^{b}, —N(R^{a})SO₂R^{b}, —SO₂NR^{a}R^{b}, —S(O)₀₋₂R^{a}, or —CH₂NR^{c}R^{d} wherein R^{c} and R^{d} are each independently H or (C₁-C₈)alkyl, or R^{c} and R^{d} taken together with the nitrogen to which they are attached form a monocyclic saturated heterocyclic group;

each R^{a} and R^{b} are each independently H, (C₁-C₈)alkyl, (C₃-C₈)cycloalkyl, aryl(C₁-C₈)alkyl, or a nitrogen protecting group; and

any alkyl, cycloalkyl, aryl or heteroaryl of R^{x} is optionally substituted with one, two, or three R² groups;

or a pharmaceutically acceptable salt thereof.

The invention further provides a composition comprising a compound of formula I and a pharmaceutically acceptable diluent or carrier. The composition can be a pharmaceutical composition, for example, a pharmaceutical composition for treating a disease, disorder, or medical condition mediated by FAAH activity. The composition can include an effective amount of at least one compound of formula I, or a pharmaceutically acceptable salt thereof, a pharmaceutically acceptable prodrug thereof, a pharmaceutically active metabolite thereof, or any combination thereof.

The composition can include an analgesic, such as an opioid or a non-steroidal anti-inflammatory drug. In some embodiments, the composition can include a second active ingredient, for example, aspirin, acetaminophen, opioids, ibuprofen, naproxen, COX-2 inhibitors, gabapentin, pregabalin, or tramadol.

The invention also provides a method for treating a subject suffering from or diagnosed with a disease, disorder, or medical condition mediated by FAAH activity. The method can include administering to a subject in need of such treatment an effective amount of at least one compound of formula I, a pharmaceutically acceptable salt thereof, a pharmaceutically acceptable prodrug thereof, or a pharmaceutically active metabolite thereof, or a composition containing said ingredient.

The disease, disorder, or medical condition can include anxiety, depression, pain, sleep disorders, eating disorders, inflammation, movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, neuroinflammation, or a combination thereof. In certain embodiments, the disease, disorder, or medical condition is anxiety, pain, inflammation, sleep disorders, eating disorders, and movement disorders.

The invention further provides a method of inhibiting fatty acid amide hydrolase activity comprising contacting the fatty acid amide hydrolase (FAAH) with an effective amount of a compound of formula I. The method can include contacting the FAAH either in vivo or in vitro.

Additionally, the invention provides intermediates for the synthesis of compounds of formula I, as well as methods of preparing compounds of formula I. The invention also provides compounds of formula I that are useful as intermediates for the synthesis of other useful compounds. The invention further provides for the use of compounds of formula I for the manufacture of medicaments useful for the treatment conditions in a mammal, such as a human, that are mediated by FAAH.

Additional embodiments, features, aspects, and advantages of the invention will be apparent from the following detailed description and through practice of the invention.

### DETAILED DESCRIPTION

The invention may be more fully appreciated by reference to the following description, including the following glossary of terms and the concluding examples. For the sake of brevity, the disclosures of the publications, including patents, cited in this specification are herein incorporated by reference. Reference is herein made to the subject matter recited by certain claims, examples of which are illustrated in the accompanying structures and formulas. While the exemplary subject matter will be described, it will be understood that the exemplary descriptions are not intended to limit the claims. On the contrary, the inventive subject matter is intended to cover all alternatives, modifications, and equivalents, which may be included within the scope of the presently disclosed subject matter as defined by the claims.

References in the specification to “an embodiment” or “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is within the knowledge of one skilled in the art to affect such feature, structure, or characteristic in connection with other embodiments whether or not explicitly described in connection with the first feature, structure, or characteristic.

### Definitions

As used herein, certain terms have the following meanings. All other terms and phrases used in this specification have their ordinary meanings as one of skill in the art would understand. Such ordinary meanings may be obtained to their use in the art and by reference to general and technical dictionaries, such as Hawley's Condensed Chemical Dictionary 14^{th} Edition, by R. J. Lewis, John Wiley & Sons, New York, N.Y., 2001; Webster's New World Dictionary, Simon & Schuster, New York, N.Y., 1995; and The American Heritage Dictionary of the English Language, Houghton Mifflin, Boston Mass., 1981.

The following explanations of certain terms are meant to be illustrative rather than exhaustive. These terms have their ordinary meanings given by usage in the art and in addition include the following explanations.

The term “and/or” refers to any one of the items, any combination of the items, or all of the items with which this term is associated.

The singular forms “a,” “an,” and “the” include plural reference unless the context clearly dictates otherwise. For example, a reference to “a compound” includes a plurality of such compounds, so that a compound X includes a plurality of compounds X. Thus, the singular article of speech forms “a,” “an,” and “the” include plural reference such as, but not limited to, multiples of the element, term, feature, compound, composition, method, and the like, to which the article of speech refers unless the context clearly dictates otherwise.

The term “about” can refer to a variation of ±5%, 10%, or 20% of the value specified. For example, “about 50” percent can in some embodiments carry a variation from 45 to 55 percent. For integer ranges, the term “about” can include one or two integers greater than and less than a recited integer.

As used herein, “contacting” refers to the act of touching, making contact, or of bringing to immediate or close proximity, including at the molecular level, such as in solution, in a tissue, or in a cell, for example, in vitro or in vivo.

As to any of the groups or “substituents” described herein, each can further include one or more (e.g., 1, 2, 3, 4, 5, or 6) substituents. It is understood, of course, that such groups do not contain any substitution or substitution patterns which are sterically impractical and/or synthetically non-feasible.

The terms “comprising”, “including”, “having”, and “composed of” are open-ended terms as used herein.

The term “alkyl” refers to a straight- or branched-chain alkyl group having from 1 to about 20 carbon atoms in the chain. Examples of alkyl groups include methyl (Me, which also may be structurally depicted by a / symbol), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “alkenyl” refers to a straight- or branched-chain alkenyl group having from 2 to 12 carbon atoms in the chain. (The double bond of the alkenyl group is formed by two sp² hybridized carbon atoms.) Illustrative alkenyl groups include prop-2-enyl, but-2-enyl, but-3-enyl, 2-methylprop-2-enyl, hex-2-enyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

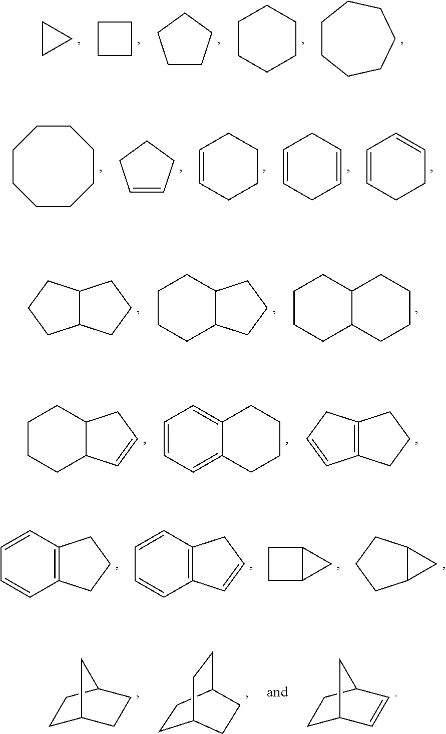

A “heterocycle” or “heterocycloalkyl” group refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative examples of heterocycle groups include the following entities, in the form of properly bonded moieties:

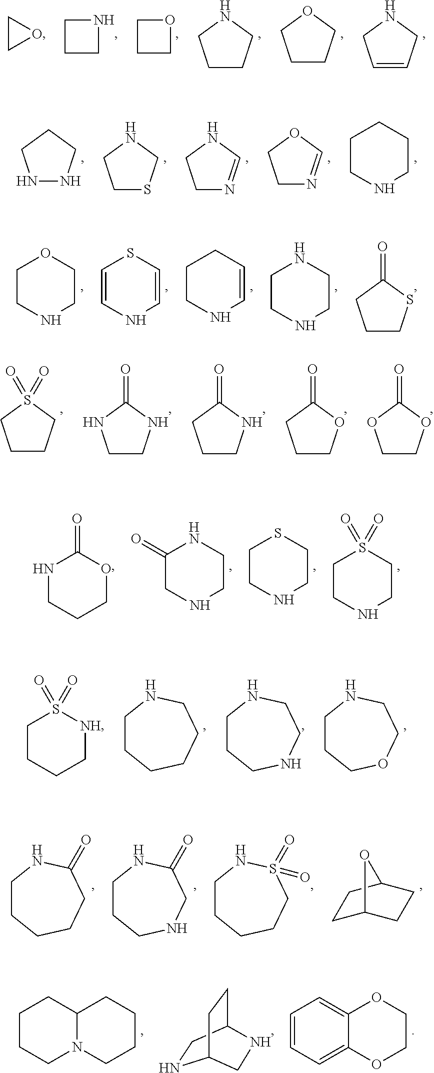

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-14 carbon atoms, about 6-13 carbon atoms, or about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted.

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

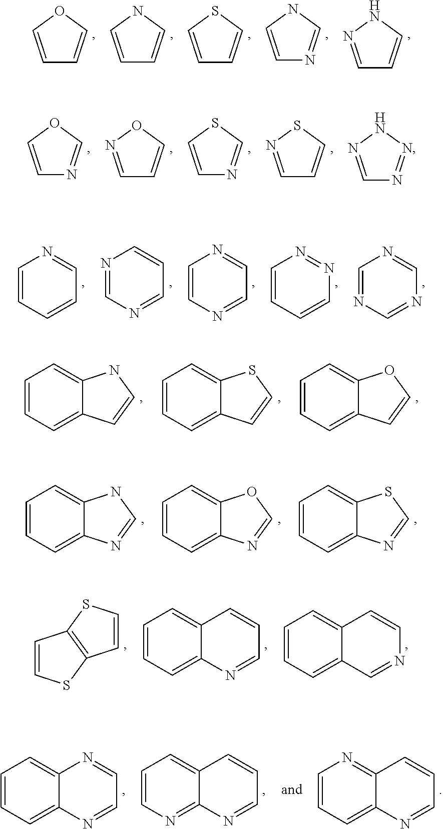

Those skilled in the art will recognize that the species of cycloalkyl, heterocycle, and heteroaryl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted in some embodiments but can be substituted in other embodiments. Suitable substituent groups include, e.g., alkyl, alkenyl, alkynyl, alkoxy, halo, haloalkyl, hydroxy, hydroxyalkyl, aryl, aroyl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl(alkyl)amine, and/or cyano. Any one or more of the aforementioned suitable substituents can also be excluded from a given embodiment, for example, a compound of any one of formulas I-IV.

Any formula given herein is intended to represent compounds having structures depicted by the structural formula as well as certain variations or forms. In particular, compounds of any formula given herein may have asymmetric centers and therefore exist in different enantiomeric and/or diastereomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to limit the definition of the moiety for the variable appearing elsewhere. In other words, where a variable appears more than once, the choice of the species from a specified list is independent of the choice of the species for the same variable elsewhere in the formula.

The invention also includes pharmaceutically acceptable salts of the compounds represented by formula I, preferably of those described above and of the specific compounds exemplified herein, and methods of treatment using such salts.

A “pharmaceutically acceptable salt” is intended to mean a salt of a free acid or base of a compound represented by formula I that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response.

A compound of formula I may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, besylates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of formula I contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of formula I includes an acid moiety, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The term “solvate” refers to a solid compound that has one or more solvent molecules associated with its solid structure. Solvates can form when a compound is crystallized from a solvent, wherein one or more solvent molecules become integral part(s) of the crystal. The compounds of formula I can be solvates, for example, ethanol solvates. Likewise, a “hydrate” refers to a solid compound that has one or more water molecules associated with its solid structure. A hydrate is a subgroup of solvates. Hydrates can form when a compound is crystallized from water, wherein one or more water molecules become integral part(s) of the crystal. The compounds of formula I can be hydrates.

### Prodrugs and Metabolites

The invention also relates to pharmaceutically acceptable prodrugs of a compound of formula I, and treatment methods employing such a pharmaceutically acceptable prodrugs. The term “prodrug” refers to a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of formula I). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of formula I. Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone, and protected versions thereof.

Additional types of a prodrug may be produced, for instance, by derivatizing free carboxyl groups of structures of formula I as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs.

Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. These prodrug moieties may incorporate groups including ether, amine, and carboxylic acid functionalities.

The present invention also relates to a pharmaceutically active metabolite of a compound of formula I, and use(s) of such a metabolite in the methods of the invention. A “pharmaceutically active metabolite” refers to a pharmacologically active product of metabolism in the body of a compound of formula I or salt thereof. A prodrug or an active metabolite of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

### Therapeutic Methods

A compound of formula I and its pharmaceutically acceptable salt, its pharmaceutically acceptable prodrug, and its pharmaceutically active metabolite (collectively, “active agents”) of the present invention can be useful as FAAH inhibitors in the methods of the invention. The active agents may be used for the treatment or prevention of medical conditions, diseases, or disorders mediated through inhibition or modulation of FAAH, such as those described herein. Active agents according to the invention may therefore be used as an analgesic, anti-depressant, cognition enhancer, neuroprotectant, sedative, appetite stimulant, or contraceptive.

Compounds and pharmaceutical compositions suitable for use in the present invention include those wherein the active agent is administered in an effective amount to achieve its intended purpose. The phrase “therapeutically effective amount” refers to an amount effective to treat the disease, disorder, and/or condition, for example, an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., to treat or prevent a disease or disorder, or to treat the symptoms of the disease or disorder, in a host.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” extend to prophylaxis and include prevent, prevention, preventing, lowering, stopping or reversing the progression or severity of the condition or symptoms being treated. As such, the term “treatment” includes both medical, therapeutic, and/or prophylactic administration, as appropriate.

Exemplary medical conditions, diseases, and disorders include anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, epilepsy, Niemann-Pick disease, Parkinson's disease, Huntington's chorea, optic neuritis, autoimmune uveitis, symptoms of drug withdrawal, nausea, emesis, sexual dysfunction, post-traumatic stress disorder, or cerebral vasospasm, or combinations thereof.

The active agents may be used to treat subjects (patients) diagnosed with or suffering from a disease, disorder, or condition mediated through FAAH activity. The term “treat” or “treating” as used herein is intended to refer to administration of an agent or composition of the invention to a subject for the purpose of effecting a therapeutic or prophylactic benefit through modulation of FAAH activity. Treating includes reversing, ameliorating, alleviating, inhibiting the progress of, lessening the severity of, or preventing a disease, disorder, or condition, or one or more symptoms of such disease, disorder or condition mediated through modulation of FAAH activity.

The term “subject” refers to a mammalian patient in need of such treatment, such as a human. “Modulators” include both inhibitors and activators, where “inhibitors” refer to compounds that decrease, prevent, inactivate, desensitize or down-regulate FAAH expression or activity, and “activators” are compounds that increase, activate, facilitate, sensitize, or up-regulate FAAH expression or activity.

Accordingly, the invention relates to methods of using the active agents described herein to treat subjects diagnosed with or suffering from a disease, disorder, or condition mediated through FAAH activity, such as anxiety, pain, sleep disorders, eating disorders, inflammation, or movement disorders (e.g., multiple sclerosis).

Symptoms or disease states are intended to be included within the scope of “medical conditions, disorders, or diseases.” For example, pain may be associated with various diseases, disorders, or conditions, and may include various etiologies. Illustrative types of pain treatable with a FAAH-modulating agent according to the invention include cancer pain, postoperative pain, GI tract pain, spinal cord injury pain, visceral hyperalgesia, thalamic pain, headache (including stress headache and migraine), low back pain, neck pain, musculoskeletal pain, peripheral neuropathic pain, central neuropathic pain, neurogenerative disorder related pain, and menstrual pain. HIV wasting syndrome includes associated symptoms such as appetite loss and nausea. Parkinson's disease includes, for example, levodopa-induced dyskinesia. Treatment of multiple sclerosis may include treatment of symptoms such as spasticity, neurogenic pain, central pain, or bladder dysfunction. Symptoms of drug withdrawal may be caused by, for example, addiction to opiates or nicotine. Nausea or emesis may be due to chemotherapy, postoperative, or opioid related causes. Treatment of sexual dysfunction may include improving libido or delaying ejaculation. Treatment of cancer may include treatment of glioma. Sleep disorders include, for example, sleep apnea, insomnia, and disorders calling for treatment with an agent having a sedative or narcotic-type effect. Eating disorders include, for example, anorexia or appetite loss associated with a disease such as cancer or HIV infection/AIDS.

### Compounds and Methods of the Invention

The invention provides useful FAAH modulators, for example, inhibitors, including compounds of formula I:

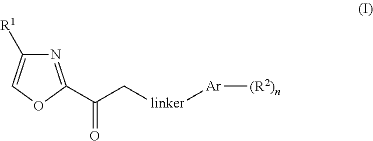

wherein

R¹ is —Y—R^{x};

Y is —CH₂—, —O—, —CH₂O—, —OCH₂—, —C(═O)—, —CO₂—, —OC(═O)—, —S—, —S(O)—, —S(O)₂—, —N(R^{a})—, or a direct bond;

R^{x} is H, halo, (C₁-C₂₀)alkyl, (C₁-C₈)cycloalkyl, trifluoromethyl, aryl, heteroaryl, —CN, —NO₂, or —NR^{a}R^{b};

linker is a (C₁-C₂₀)alkyl chain wherein one to five carbons of the chain are optionally be replaced with O or S, or linker is a direct bond;

Ar is (C₆-C₁₄)aryl;

each R² is independently H, —X—R³, or —X-Ph-X—R³;

n is 1, 2, 3, or 4;

each X is independently —CH₂—, —O—, —CH₂O—, —OCH₂—, —C(═O)—, —CO₂—, —OC(═O)—, —S—, —S(O)—, —S(O)₂—, —N(R^{a})—, or a direct bond;

each R³ is independently H, (C₁-C₈)alkyl, (C₃-C₈)cycloalkyl, aryl, heteroaryl, —CF₃, —CN, —C(O)(C₁-C₈)alkyl optionally substituted with one, two, or three fluoro substituents, —C₂(C₁-C₈)alkyl, —CO₂H, —C(O)NR^{a}R^{b}, —OH, —O(C₁-C₈)alkyl, -halo, —NO₂, —NR^{a}R^{b}, —N(R^{a})C(O)R^{b}, —N(R^{a})SO₂R^{b}, —SO₂NR^{a}R^{b}, —S(O)₀₋₂R^{a}, or —CH₂NR^{c}R^{d} wherein R^{c} and R^{d} are each independently H or (C₁-C₈)alkyl, or R^{c} and R^{d} taken together with the nitrogen to which they are attached form a monocyclic saturated heterocyclic group;

each R^{a} and R^{b} are each independently H, (C₁-C₈)alkyl, (C₃-C₈)cycloalkyl, aryl(C₁-C₈)alkyl, or a nitrogen protecting group; and

any alkyl, cycloalkyl, aryl or heteroaryl of R^{x} is optionally substituted with one, two, or three R² groups;

or a pharmaceutically acceptable salt thereof.

In certain embodiments, the compound of formula I can be a compound of formula II:

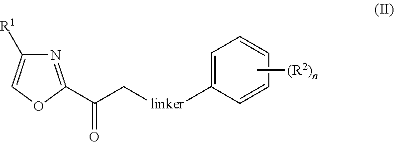

wherein R¹, linker, and R² are as defined for formula I.

In one embodiment, the compound of formula I can be a compound of formula III:

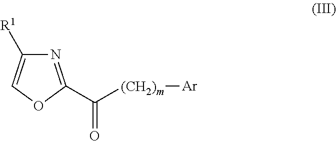

wherein R¹ and Ar are as defined for formula I, and wherein m is 1 to about 20, for example, about 2 to about 10, wherein one to five carbons of the chain can optionally be replaced with one or more O or S atoms. In certain embodiments, m can be 1, 2, 3, 4, 5, 6, 7, 8, 9, 10, 11, 12, 13, 14, 15, 16, 17, 18, 19, or 20, or any range between any two of the foregoing integers.

In certain embodiments, the compound of formula I can be a compound of formula IV:

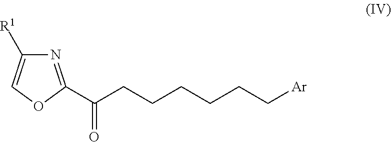

wherein R¹ and Ar as defined for formula I. In some embodiments, Ar can be a phenyl or naphthyl group, optionally substituted with 1, 2, 3, 4, or 5 substituents, as defined herein.

In yet another embodiment, the compound of formula I can be a compound of formula V:

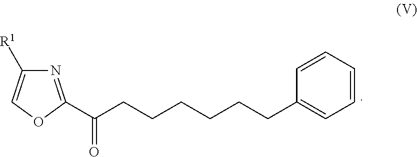

In yet another embodiment, a compound of the invention includes a compound of formula VI:

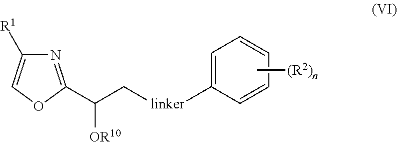

wherein R¹⁰ H or an oxygen protecting group, such as a silicon protecting group, for example, TBS, TIPS or TBDPS.

In one embodiment, R¹ can be H, halo, (C₁-C₆)alkyl, (C₁-C₆)alkoxy, (C₁-C₆)alkylthio, —CHO, carboxy, (C₁-C₆)alkanoyl, (C₁-C₆)alkoxycarbonyl, trifluoromethyl, trifluoromethoxy, phenyl, pyridyl, —CN, or —C(═O)—NR^{a}R^{b}. In another embodiment, R¹ is fluoro, chloro, iodo, methyl, ethyl, propyl, —OMe, —OEt, —SMe, —SEt, —C(═O)Me, —CO₂Me, —CONH₂, —CONH(Me) (N-methyl carbamide), or —CON(Me)₂, (N,N-dimethyl carbamide).

In one embodiment, linker is a (C₁-C₈)alkyl or a direct bond. In another embodiment, linker can be a carbon chain of 1, 2, 3, 4, 5, 6, 7, 8, 9, 10, 11, 12, 13, 14, 15, 16, 17, 18, 19, or 20 carbon atoms, or any range between any two of the foregoing integers. One to about five carbons of the chain can optionally be replaced with O or S atoms.

In one embodiment, R² is H and n is 1.

In another embodiment, R₂ is —X—R³; X is —O—, —S—, or a direct bond; and R³ is phenyl.

In one embodiment, the heteroaryl group can be selected from the following:

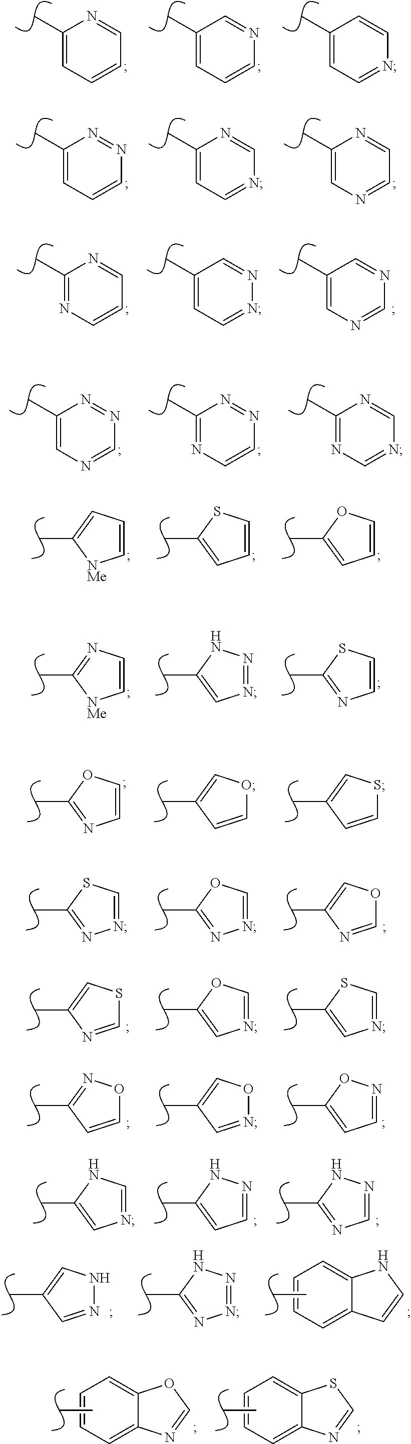

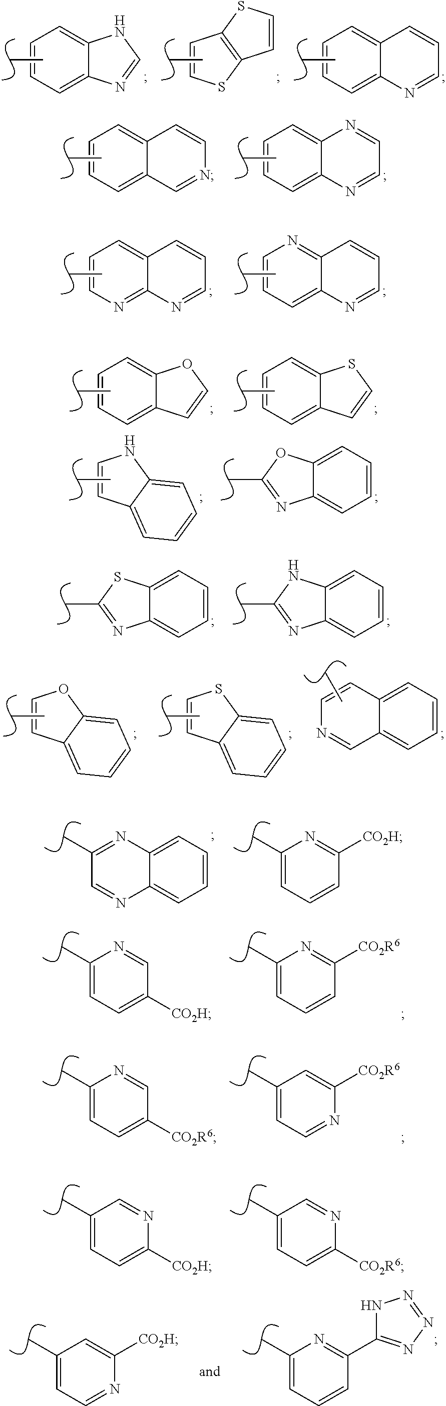

where R⁶ can be (C₁-C₆)alkyl. Any aryl or heteroaryl can be optionally substituted with one or more R² groups, wherein each R² is independently H, —X—R³, or —X-Ph-X—R³; and X and R³ are as defined above for formula I.

In one embodiment, R^{c} and R^{d} are taken together with the nitrogen to which they are attached to form a piperidine, morpholine, thiomorpholine, pyrrolidine, or N-methylpiperazine group.

In one embodiment, the invention provides a compound selected from 1-(4-bromooxazol-2-yl)-7-phenylheptan-1-one; 1-(4-chlorooxazol-2-yl)-7-phenylheptan-1-one; 1-(4-iodooxazol-2-yl)-7-phenylheptan-1-one; 1-(4-methyloxazol-2-yl)-7-phenylheptan-1-one; 1-(4-(methylthio)oxazol-2-yl)-7-phenylheptan-1-ol; 1-(4-(methylthio)oxazol-2-yl)-7-phenylheptan-1-one; 2-(7-phenylheptanoyl)oxazole-4-carbaldehyde; 1-(4-acetyloxazol-2-yl)-7-phenylheptan-1-one; 7-phenyl-1-(4-(2,2,2-trifluoroacetypoxazol-2-yl)heptan-1-one; methyl 2-(7-phenylheptanoyl)oxazole-4-carboxylate; 7-phenyl-1-(4-(pyridin-2-yl)oxazol-2-yl)heptan-1-one; 7-phenyl-1-(4-(pyridin-3-yl)oxazol-2-yl)heptan-1-one; 7-phenyl-1-(4-(pyridin-4-yl)oxazol-2-yl)heptan-1-one; 7-phenyl-1-(4-phenyloxazol-2-yl)heptan-1-one; 2-(7-phenylheptanoyl)oxazole-4-carboxylic acid; 2-(7-phenylheptanoyl)oxazole-4-carboxamide; N-methyl-2-(7-phenylheptanoyl)oxazole-4-carboxamide; N,N-dimethyl-2-(7-phenylheptanoyl)oxazole-4-carboxamide; 2-(7-phenylheptanoyl)oxazole-4-carbonitrile; 7-phenyl-1-(4-(trifluoromethyl)oxazol-2-yl)heptan-1-one; or 1-(4-methoxyoxazol-2-yl)-7-phenylheptan-1-one; or a pharmaceutically acceptable salt, solvate, prodrug, metabolite, or hemiketal thereof, or a composition thereof.

The invention also provides a composition comprising a compound of any one of formulas I-V and a pharmaceutically acceptable diluent or carrier. The composition can be a pharmaceutical composition. The pharmaceutical composition can include an analgesic, such as an opioid or a non-steroidal anti-inflammatory drug. Examples of such analgesics include aspirin, acetaminophen, opioids, ibuprofen, naproxen, COX-2 inhibitors, gabapentin, pregabalin, tramadol, or combinations thereof.

Accordingly, the invention also provides a method of treating a subject suffering from or diagnosed with a disease, disorder, or medical condition mediated by FAAH activity, comprising administering to the subject in need of such treatment an effective amount of at least one compound of formula I, a pharmaceutically acceptable salt thereof, a pharmaceutically acceptable prodrug thereof, or a pharmaceutically active metabolite thereof. The disease, disorder, or medical condition can include anxiety, depression, pain, sleep disorders, eating disorders, inflammation, movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, neuroinflammation, or a combination thereof.

The invention further includes a pharmaceutical composition for treating a disease, disorder, or medical condition mediated by FAAH activity, comprising: (a) an effective amount of at least one compound of formula I, or a pharmaceutically acceptable salt, a pharmaceutically acceptable prodrug, or an pharmaceutically active metabolite thereof, or any combination thereof, and a pharmaceutically acceptable excipient. The invention also includes a method of inhibiting fatty acid amide hydrolase activity comprising contacting the FAAH with an effective amount of a compound of any one of formulas I-V.

### Protecting Groups

The term “protecting group” refers to any group that, when bound to a hydroxyl, nitrogen, or other heteroatom prevents undesired reactions from occurring at this group and that can be removed by conventional chemical or enzymatic steps to reestablish the ‘unprotected’ hydroxyl, nitrogen, or other heteroatom group. The particular removable group employed is often interchangeable with other groups in various synthetic routes. Certain removable protecting groups include conventional substituents such as, for example, allyl, benzyl, acetyl, chloroacetyl, thiobenzyl, benzylidine, phenacyl, methyl methoxy, silyl ethers (e.g., trimethylsilyl (TMS), t-butyl-diphenylsilyl (TBDPS), triisopropylsilyl (TIPS), or t-butyldimethylsilyl (TBS)) and any other group that can be introduced chemically onto a hydroxyl functionality and later selectively removed either by chemical or enzymatic methods in mild conditions compatible with the nature of the product.

A large number of protecting groups and corresponding chemical cleavage reactions are described in Protective Groups in Organic Synthesis, Theodora W. Greene (John Wiley & Sons, Inc., New York, 1991, ISBN 0-471-62301-6) (“Greene”, which is incorporated herein by reference in its entirety). Greene describes many nitrogen protecting groups, for example, amide-forming groups. In particular, see Chapter 1, Protecting Groups: An Overview, pages 1-20, Chapter 2, Hydroxyl Protecting Groups, pages 21-94, Chapter 4, Carboxyl Protecting Groups, pages 118-154, and Chapter 5, Carbonyl Protecting Groups, pages 155-184. See also Kocienski, Philip J.; Protecting Groups (Georg Thieme Verlag Stuttgart, New York, 1994), which is incorporated herein by reference in its entirety. Some specific protecting groups that can be employed in conjunction with the methods of the invention are discussed below.

Typical nitrogen and oxygen protecting groups described in Greene (pages 14-118) include benzyl ethers, silyl ethers, esters including sulfonic acid esters, carbonates, sulfates, and sulfonates. For example, suitable nitrogen or oxygen protecting groups can include substituted methyl ethers; substituted ethyl ethers; p-chlorophenyl, p-methoxyphenyl, 2,4-dinitrophenyl, benzyl; substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, diphenylmethyl, 5-dibenzosuberyl, triphenylmethyl, p-methoxyphenyl-diphenylmethyl, di(p-methoxyphenyl)phenylmethyl, tri(p-methoxyphenyl)methyl, 1,3-benzodithiolan-2-yl, benzisothiazolyl S,S-dioxido); silyl ethers (silyloxy groups) (trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxy-phenylsilyl); esters(formate, benzoylformate, acetate, choroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate(levulinate), pivaloate, adamantoate, crotonate, 4-methoxycrotonate, benzoate, p-phenylbenzoate, 2,4,6-trimethylbenzoate(mesitoate)); carbonates(methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, allyl, p-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate); groups with assisted cleavage (2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate, 4-(methylthiomethoxy)butyrate, miscellaneous esters (2,6-dichloro-4-methylphenoxyacetate, 2,6-dichloro-4-(1,1,3,3 tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate(tigloate), o-(methoxycarbonyl)benzoate, p-poly-benzoate, α-naphthoate, nitrate, alkyl N,N,N′,N′-tetramethyl-phosphorodiamidate, n-phenylcarbamate, borate, 2,4-dinitrophenylsulfenate); and sulfonates (sulfate, methanesulfonate(mesylate), benzylsulfonate, tosylate, triflate).

Delivery Modes and Preparations therefor

In treatment methods according to the invention, an effective amount of at least one active agent is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

An exemplary dose can be in the range of from about 0.001 to about 200 mg of active agent per kg of subject's body weight per day, preferably about 0.05 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day. Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be co-administered separately with an active agent of formula I or included with such an agent in a pharmaceutical composition according to the invention. In an example of an embodiment, additional active ingredients are those that are known or discovered to be effective in the treatment of conditions, disorders, or diseases mediated by FAAH activity, such as another FAAH modulator or a compound active against another target associated with the particular condition, disorder, or disease. The combination may serve to increase efficacy (e.g., by including in the combination a compound potentiating the potency or effectiveness of an active agent according to the invention), decrease one or more side effects, or decrease the required dose of the active agent according to the invention. In one illustrative embodiment, a composition may contain one or more additional active ingredients, for example, one or more of opioids, NSAIDs (e.g., ibuprofen, cyclooxygenase-2 (COX-2) inhibitors, and naproxen), gabapentin, pregabalin, tramadol, acetaminophen, and/or aspirin.

The active agents of the invention can be used, alone or in combination with one or more additional active ingredients, to formulate pharmaceutical compositions of the invention. A pharmaceutical composition of the invention can include, for example, (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the methods by a suitable route of delivery, e.g., oral, parenteral, rectal, topical, or ocular routes, or by inhalation. Suitable routes include administration by catheter or by injection (e.g., IV, IM, or SC).

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 0.05 to about 50 mg/kg daily, or from about 0.05 to about 20 mg/kg daily, or from about 0.1 to about 10 mg/kg daily. These dosages may be orally administered using any of the foregoing preparations and the administration will be accomplished according to the wisdom and judgment of the patient's attending physician.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days. Administration will be accomplished according to the wisdom and judgment of the patient's attending physician.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

### Compound Preparation and Enzyme Inhibitory Activity

Exemplary chemical entities useful in methods of the invention are described herein by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified or defined, the variables are as defined above in reference to formula I.

Compounds of formula I can be prepared by metallation of the 2-position of substituted oxazoles and reaction with suitable acid chlorides (see Harn et al., Tetrahedron Lett. 1995, 36, 9453-9456). Alternatively, compounds of formula I can be prepared by metallation of oxazole and reaction with suitable aldehydes to form alcohols. Protection of the alcohol functionality with a suitable protecting group, PG (such as a silyl protecting group), provides compounds that can be metallated at the 5-position. Metallation of the 5-position of the oxazole, followed by bromination and a subsequent halogen dance reaction provides the C-4 bromide. Palladium-mediated coupling with suitable reagents R¹-M, where M is —SnBu₃, —B(OH)₂, I, or Br, followed by deprotection of the alcohol protecting group and oxidation under standard conditions, provides compounds of formula I. For suitable and related synthetic techniques, see Boger et al. J. Med. Chem. 2005, 48, 1849-1856.

Compounds of formula I may be converted to their corresponding salts using methods described in the art. In particular, an amine-containing compound of formula I may be treated with trifluoroacetic acid, HCl, or citric acid in a solvent such as Et₂O, CH₂Cl₂, THF, and MeOH to provide the corresponding salt form.

Compounds prepared according to the schemes described below may be obtained as single enantiomers, diastereomers, or regioisomers, by enantio-, diastero-, or regiospecific synthesis, or by resolution. Compounds prepared according to the schemes above may alternately be obtained as racemic (1:1) or scalemic (non-racemic) (not 1:1) mixtures or as mixtures of diastereomers or regioisomers. Where racemic and scalemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

Fatty acid amide hydrolase (FAAH) is the enzyme that serves to hydrolyze endogenous lipid amides including anandamide (1a) and oleamide (1b) (Scheme 1). Its distribution is consistent with its role in degrading and regulating such neuromodulating and signaling fatty acid amides at their sites of action. Although it is a member of the amidase signature family of serine hydrolases, for which there are a number of prokaryotic enzymes, it is currently the only characterized mammalian enzyme bearing the family's unusual Ser-Ser-Lys catalytic triad.

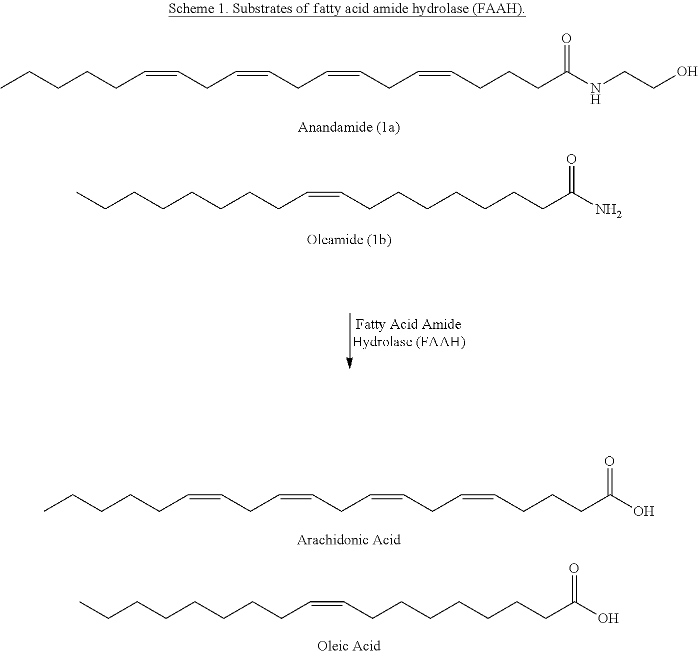

Due to the therapeutic potential of inhibiting FAAH, especially for the treatment of pain, inflammatory, or sleep disorders, there has been an increasing interest in the development of selective and potent inhibitors of the enzyme. Early studies shortly following the initial characterization of the enzyme led to the discovery that the endogenous sleep-inducing molecule 2-octyl α-bromoacetoacetate is an effective FAAH inhibitor. A series of nonselective reversible inhibitors bearing an electrophilic ketone (e.g., trifluoromethyl ketone-based inhibitors) and a set of irreversible inhibitors (e.g., fluorophosphonates and sulfonyl fluorides) were also reported. To date, only two classes of inhibitors have been disclosed that provide opportunities for the development of inhibitors with therapeutic potential.

One class is the reactive aryl carbamates and ureas that irreversibly acylate a FAAH active site serine. A second class is the α-ketoheterocycle-based inhibitors that bind to FAAH via reversible hemiketal formation with an active site serine. Many of these latter competitive inhibitors are not only potent and extraordinarily selective for FAAH versus other mammalian serine hydrolases, but members of this class have been shown to be efficacious analgesics in vivo.

Compounds of the invention bearing varied C4 oxazole substituents were prepared from the readily available oxazole 2 (Kimball et al., J. Med. Chem. 2008, 51, 937), enlisting its in situ conversion to the isomeric 4-bromooxazole via a halogen dance rearrangement; Scheme 2. Thus, C4-lithiation of 2 followed by its in situ rearrangement to the more stable 5-lithio-4-bromooxazole and its quench with water provided 3b, a useful precursor for numerous derivatives. A series of derivatives was accessed by metallation of 3b with n-BuLi or t-BuLi followed by reaction with appropriate electrophiles (NCS, I₂, CH₃I, (MeS)₂, DMF, CH₃CONMe₂, CF₃CO₂Et, NCCO₂Me).

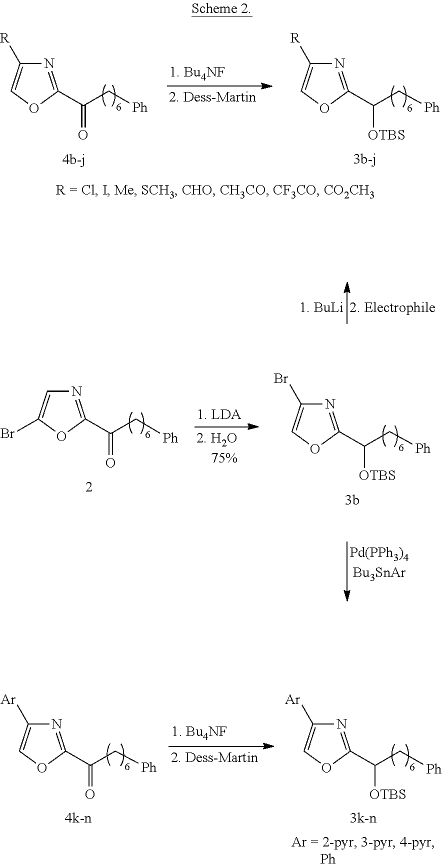

Similarly, the C4 pyridine (3k, 3l, and 3m) and phenyl (3n) derivatives were prepared from bromide 3b using a Stille coupling reaction with the respective pyridyl or phenyl tributylstannanes. Many of these derivatives served as precursors to additional compounds of the invention bearing further modified C4 substituents.

Methyl ester 4j was directly converted to its corresponding carboxylic acid 4o and carboxamide 4p using LiOH and methanolic ammonia, respectively; Scheme 3. Carboxylic acid 4o was also coupled with methylamine and dimethylamine to give the substituted carboxamides 4q and 4r. Carboxamide 4p was dehydrated with TFAA and pyridine to provide nitrile 4s. The trifluoromethyl derivative 3t was prepared from iodide 3d using the method developed by Chen et al. (see J. Chem. Soc., Perkin Trans. 1 1997, 3053) and iodide 3d also served as the precursor to methyl ether 3u. In each case, deprotection of the TBS ether followed by Dess-Martin periodinane oxidation of the liberated alcohol yielded the corresponding α-ketooxazole (4b-u); Schemes 2 and 3.

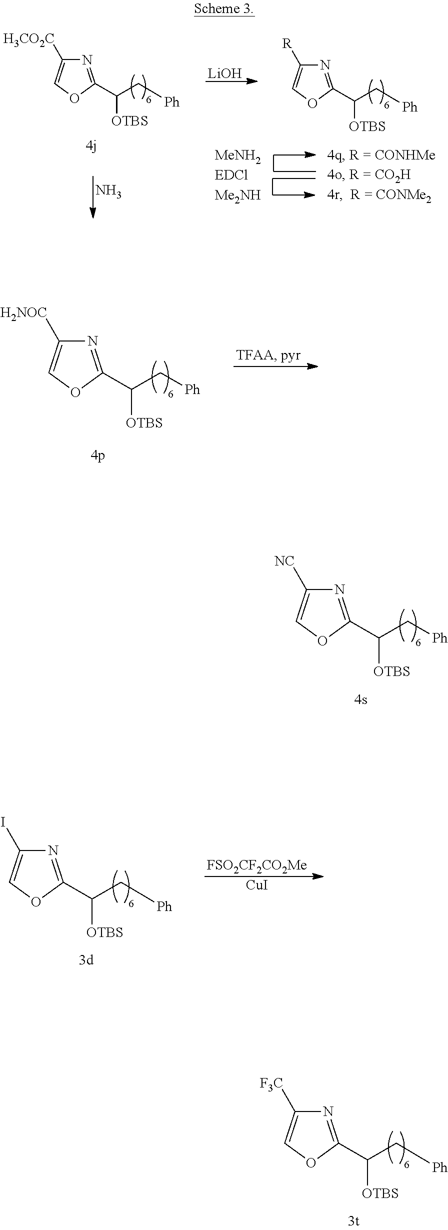

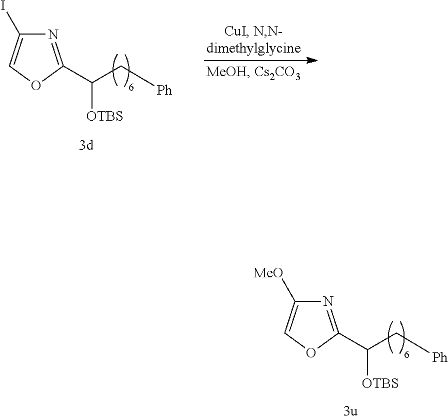

The FAAH inhibition derived from the examination of a series of inhibitors is provided in Table 1. The C4 substituted oxazole compounds provided significant inhibition of rat FAAH, including inhibition at levels as low as 0.5 nM.

TABLE 1
Rat FAAH inhibition (Kᵢ, nM).

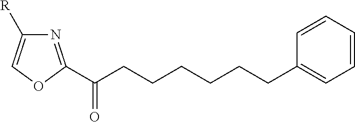

| cmpd | R | Kᵢ, nM | cmpd | R | Kᵢ, nM |
|---|---|---|---|---|---|
| 4a | H | 48 | 4k | 2-Pyr | 1.9 |
| 4b | Br | 3.0 | 4l | 3-pyr | 18 |
| 4c | Cl | 4.0 | 4m | 4-pyr | 1.6 |
| 4d | I | 6.5 | 4n | Ph | 65 |
| 4e | CH₃ | 520 | 4o | CO₂H | 53 |
| 4f | SCH₃ | 29 | 4p | CONH₂ | 1.6 |
| 4g | CHO | 55 | 4q | CONHMe | 1.8 |
| 4h | COCH₃ | 2.0 | 4r | CONMe₂ | 35 |
| 4i | CF₃CO | 470 | 4s | CN | 0.5 |
| 4j | CO₂CH₃ | 3.4 | 4t | CF₃ | 3.7 |
| | | | 4u | OMe | 740 |

The evaluation of Kᵢ provides information about active site binding. For example, the data indicates that 4o binds the FAAH active site as its deprotonated carboxylate as opposed to its carboxylic acid, in view of the measured Kᵢ and given the pH of the enzyme assay conditions (pH=9.0). More subtly, aldehyde 4g (and trifluoromethyl ketone 4i) was established to exist in protic solution as gem diols (at C4, not C2; ¹H and ¹³C NMR, data in Examples below).

It was also determined that compounds 4g and 4i inhibit FAAH with potencies at a level more consistent with this C4 substituent gem diol versus carbonyl active site binding, although the latter C(OH)₂CF₃ gem diol most likely suffers significant destabilizing steric interactions at the enzyme active site comparable to that of a t-butyl substituent.

Several inhibitors were surprisingly potent, for example, inhibitors 4m, 4k, 4p, and 4q. All four may benefit from additional H-bonding at the active site, which increases affinity beyond that of some other derivatives. Based on their relative Kᵢ's, the 4-pyridyl derivative 4m and, to a lesser extent, the 2-pyridyl derivative 4k may interact with the catalytic Lys142 at the FAAH active site where such a potential H-bond may be regarded not only as a conventional H-bond stabilizing interaction, but also as a partial protonation of the pyridyl nitrogen, enhancing its electron-withdrawing properties. Similarly, the primary carboxamide 4p and, to a lesser extent, the secondary carboxamide 4q provided significant and surprisingly effective inhibitory properties.

It is theorized that this behavior is the result of a productive H-bonding interaction of RCONHR at the FAAH active site for 4p and 4q (but not 4r) that further increases affinity, and/or destabilizing steric interactions that emerge only with the tertiary amide 4r. Two substituents (-Me, -OMe) provide somewhat lower potency that others. Both may represent electron-donating and electron-rich substituents, which may lower the inhibitory activity. Thus, while additional substituent features can modulate the binding affinity of compounds of the invention (e.g., H-bonding, hydrophobic or steric interactions), the electronic effect of the substituent can also provide an effect on inhibitory activity.

Finally, the oxazole C4 substituents in the inhibitors disclosed herein not only influence the FAAH inhibitor potency, but they can have an equally remarkable impact on the FAAH inhibition selectivity. There are no other characterized mammalian members of the serine hydrolase family that bear the amidase signature sequence and its unusual Ser-Ser-Lys catalytic triad, and no resulting close family of enzymes against which to counter screen inhibitory compounds. However, a proteome-wide assay capable of simultaneously interrogating all mammalian serine hydrolases applicable to assessing the selectivity of reversible enzyme inhibitors was developed.

This assay, which requires no modification of the inhibitor, no purified protein for conventional substrate assay, no knowledge of candidate off-site targets or even the function or substrate of the enzymes, can globally detect, identify, and quantitate potential competitive enzyme targets in the human proteome for such inhibitors (Leung et al., Nature Biotech. 2003, 21, 687). To date, two enzymes have emerged at potential competitive targets for inhibitors in this class: triacylglycerol hydrolase (TGH) and a membrane-associated hydrolase (KIAA1363) (see Kidd et al., Biochemistry 2001, 40, 4005). Enlisting this proteome selectivity assay, inhibitors for both FAAH potency and selectivity have been able to be simultaneously discovered, identifying features of compounds that can increase binding at the FAAH active site while simultaneously disrupting KIAA1363 and TGH affinity.

This multidimensional SAR study is well highlighted by the inhibitors 4t, 4s, 4k, 4m and 4o, with results summarized in Table 2. The addition of a 4-substituent to 4a enhances the FAAH versus KIAA1363 selectivity (>25-fold selective vs 8-fold for 4a), where the 5-substituted oxazole inhibitors typically fail to inhibit KIAA1363. Similarly, the addition of a 4-substituent converts the TGH selective inhibitor 4a (>100-fold selective for TGH vs FAAH) into inhibitors that are modestly selective for FAAH (up to 5-fold selective). The exception to this is 4k, which like OL-135 (5c) was found to be >300-fold selective for FAAH versus TGH. The enhancement in FAAH selectivity (typically >100-fold, but >40,000 for 4k) is significant and illustrates the importance of C4 substituted oxazoles.

TABLE 2
Selectivity Screening, IC₅₀, μM (selectivity).

| | | | | |
|---|---|---|---|---|
| | FAAH | FAAH | KIAA1363 | TGH |
| Cmpd | Kᵢ, nM | IC₅₀, μM | IC₅₀, μM | IC₅₀, μM |

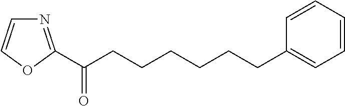

| | | | | |
|---|---|---|---|---|
| 4a (H) | 48 | 2.5 | 20 (8) | 0.02 (0.008) |

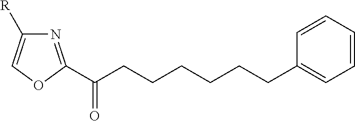

| | | | | |
|---|---|---|---|---|
| 4t (CF₃) | 3.7 | 0.4 | 10 (25) | 0.06 (0.15) |
| 4s (CN) | 0.6 | 0.02 | 30 (1500) | 0.03 (1.5) |
| 4k (2-pyr) | 1.9 | 0.03 | >100 (>3000) | 10 (330) |
| 4m (4-pyr) | 1.7 | 0.14 | >100 (>700) | 0.7 (5) |
| 4o (CO₂H) | 53 | 0.5 | >100 (>200) | 2.5 (5) |

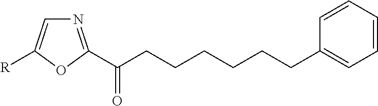

| | | | | |
|---|---|---|---|---|
| 5a (CF₃) | 0.8 | 0.07 | >100 (>1400) | 0.2 (3) |
| 5b (CN) | 0.4 | 0.007 | >100 (>14000) | 0.02 (3) |
| 5c (2-pyr) | 4.7 | 0.002 | >100 (>50000) | 0.6 (300) |
| 5d (CO₂H) | 30 | 0.9 | >100 (>100) | >100 (>100) |

As discussed above, it was found that a 4-substituent on the oxazole can enhance FAAH inhibitor potency, but oxazole inhibitors bearing both a 4-substituent and a 5-substituent were significantly less active. Although not a limitation of this disclosure, it is theorized that the two (C4 and C5 substituted) classes of oxazole-based inhibitors may bind at the FAAH active site in a manner that places the substituent in a comparable location, such as by a rotation of the oxazole orientation at the active site, reversing the location of the N and O of the heterocycle (Scheme 4). Accordingly, there may be space for one, but not two such substituents (C4 and CS) on the oxazole ring of the inhibitors disclosed herein.

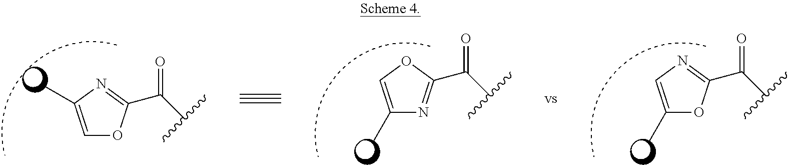

In summary, a series of C4 substituted α-ketooxazoles were examined as inhibitors of the serine hydrolase fatty acid amide hydrolase. The disclosure herein provides a useful class of potent and selective FAAH inhibitors. Experimental details on the preparation and characterization of certain inhibitors, the FAAH inhibition assay, and FAAH assay measurement errors are provided in the Examples below.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Preparation of α-Ketoheterocycle Enzyme Inhibitors

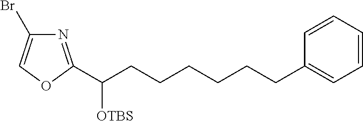

4-Bromo-2-(1-(tert-butyldimethylsilyloxy)-7-phenylheptyl)oxazole (3b). A freshly prepared solution of LDA (1.5 equiv) in THF (0.5 M) was added to a solution of 5-bromo-2-(1-(tert-butyldimethylsilyloxy)-7-phenylheptyl)oxazole (865 mg, 1 equiv) in THF (0.1 M) at −78° C. After 30 min, wet THF was added and the reaction mixture was allowed to warm to room temperature. The reaction mixture was diluted with EtOAc and extracted with saturated aqueous NaHCO₃ and saturated aqueous NaCl and dried over Na₂SO₄. Column chromatography (SiO₂, 0-5% EtOAc/hexanes) yielded 3b as a colorless oil (649 mg, 75%): ¹H NMR (600 MHz, CDCl₃) δ 7.58 (s, 1H), 7.29-7.26 (m, 2H), 7.19-7.16 (m, 3H), 4.75 (t, 1H, J=6.0 Hz), 2.59 (t, 2H, J=7.9 Hz), 1.87-1.77 (m, 2H), 1.62-1.59 (m, 2H), 1.43-1.24 (m, 6H), 0.87 (s, 9H), 0.07 (s, 3H), −0.03 (s, 3H); ¹³C NMR (150 MHz, CDCl₃) δ 166.0, 142.9, 136.7, 128.5 (2C), 128.4 (2C), 125.7, 115.4, 68.6, 36.4, 36.0, 31.5, 29.2, 25.8 (3C), 25.1, 18.3, −4.9, −5.0; ESI-TOF m/z 452.1603 (M+H⁺, C₂₂H₃₄BrNO₂Si requires 452.1615).

General Procedure A. The TBS ether (1 equiv) was dissolved in THF (0.08 M), treated with Bu₄NF (1 M in THF, 1.3 equiv) and the mixture was stirred at room temperature for 30 min under Ar. The reaction mixture was diluted with toluene and the majority of the THF was removed under reduced pressure. The remaining toluene mixture was applied directly to a silica gel column and eluted to provide the corresponding alcohol.

General Procedure B. The alcohol (1 equiv) was dissolved in CH₂Cl₂ (0.08 M) and Dess-Martin periodinane (1.5 equiv) was added. The mixture was stirred at room temperature for 1 h before being applied directly to a silica gel column and eluted to yield the pure α-ketoheterocycle.

General Procedure C. 4-Bromo-2-(1-(tert-butyldimethylsilyloxy)-7-phenylheptyl)oxazole (1 equiv) was dissolved in THF (0.1 M) at −78° C. t-BuLi (1.51 M solution in pentane, 2 equiv) was added and reaction was stirred for 90 s before the addition of the indicated electrophile (2-5 equiv). The reaction mixture was allowed to warm to room temperature and was then diluted with EtOAc and subsequently extracted with saturated aqueous NaCl and dried over Na₂SO₄. Evaporation in vacuo yielded the crude product which was purified using preparative TLC or flash chromatography (SiO₂).

General Procedure D. 4-Bromo-2-(1-(tert-butyldimethylsilyloxy)-7-phenylheptyl)oxazole (1 equiv) was dissolved in THF (0.1 M) at −78° C. t-BuLi (1.51 M solution in pentane, 2 equiv) or n-BuLi (1.1 equiv) was added and the reaction mixture was stirred for 90 s before the addition of the trifluoroethyl acetate, N,N-dimethylacetamide, or N,N-dimethylformamide (2 equiv). The reaction mixture was warmed to room temperature, followed by the addition of water and aqueous 2 N HCl. The mixture was extracted with EtOAc, washed with saturated aqueous NaCl and dried over Na₂SO₄. Evaporation in vacuo yielded the crude product which was purified using preparative TLC or flash chromatography (SiO₂).

General Procedure E. 4-Bromo-2-(1-(tert-butyldimethylsilyloxy)-7-phenylheptyl)oxazole (1 equiv), Pd(PPh₃)₄ (0.1 equiv), and the indicated aryl tributylstannane (1.5 equiv) were dissolved in anhydrous 1,4-dioxane or toluene (0.15 M) and the mixture was warmed at reflux for 24 h under Ar. The mixture was diluted with EtOAc, washed with saturated aqueous NaCl and dried over Na₂SO₄. Evaporation in vacuo yielded crude product that was purified by column chromatography (SiO₂).

General Procedure F. 2-(7-Phenylheptanoyl)oxazole-4-carboxylic acid (4o, 1 equiv), EDCI (2 equiv), and HOAt (2 equiv) were dissolved in DMF (0.1 M). The reaction mixture was cooled to 0° C. and stirred for 10 min before the amine (1.5 equiv) was added. The reaction mixture was stirred for 16 h under Ar at room temperature, diluted with H₂O and made acidic with the addition of aqueous 1 N HCl. The solution was extracted with EtOAc (2×) and the organic layers were combined, washed with saturated aqueous NaCl and dried over Na₂SO₄. Evaporation in vacuo yielded the crude amide which was purified by chromatography (SiO₂).

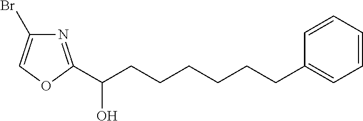

1-(4-Bromooxazol-2-yl)-7-phenylheptan-1-ol (6b). Compound 6b was prepared from 3b (22 mg) following general procedure A. Flash chromatography (20% EtOAc/hexanes) yielded 6b as a colorless oil (14 mg, 84%): ¹H NMR (600 MHz, CDCl₃) δ 7.60 (s, 1H), 7.29-7.26 (m, 2H), 7.19-7.16 (m, 3H), 4.79-4.74 (m, 1H), 2.65 (d, 1H, J=6.0 Hz), 2.60 (t, 2H, J=7.9 Hz), 1.96-1.82 (m, 2H), 1.64-1.61 (m, 3H), 1.44-1.35 (m, 4H); ¹³C NMR (150 MHz, CDCl₃) δ 166.3, 142.8, 137.1, 128.5 (2C), 128.4 (2C), 150.7, 115.6, 67.8, 36.0, 35.5, 31.5, 29.2, 24.9; ESI-TOF m/z 338.0749 (M+H⁺, C₁₆H₂₀BrNO₂ requires 338.0750).

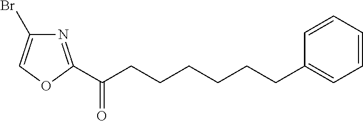

1-(4-Bromooxazol-2-yl)-7-phenylheptan-1-one. Compound 4b was prepared from 6b (13 mg) following general procedure B. Flash chromatography (15% EtOAc/hexanes) yielded 4b as a colorless oil (10 mg, 81%): ¹HNMR (600 MHz, CDCl₃) δ 7.80 (s, 1H), 7.29-7.26 (m, 2H), 7.19-7.16 (m, 3H), 3.05 (t, 2H, J=7.4 Hz), 2.61 (t, 2H, J=7.6 Hz), 1.77-1.72 (m, 2H), 1.66-1.61 (m, 2H), 1.44-1.35 (m, 4H); ¹³C NMR (150 MHz, CDCl₃) δ 187.6, 157.4, 142.8, 140.1, 128.5 (2C), 128.4 (2C), 125.8, 117.4, 39.2, 36.0, 31.4, 29.1, 29.0, 23.7; ESI-TOF m/z 336.0589 (M+H⁺, C₁₆H₁₈BrNO₂ requires 336.0594).

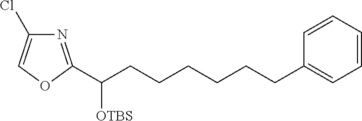

2-(1-(tert-Butyldimethylsilyloxy)-7-phenylheptyl)-4-chlorooxazole (3c). Compound 3c was prepared from 3b (37 mg) following general procedure C using N-chlorosuccinimide (NCS, 33 mg, 3 equiv) in THF as the electrophile. Preparative TLC (10% EtOAc/hexanes) yielded 3c as a colorless oil (14 mg, 43%): ¹H NMR (600 MHz, CDCl₃) δ 7.54 (s, 1H), 7.29-7.26 (m, 2H), 7.19-7.16 (m, 3H), 4.73 (t, 1H, J=6.0 Hz), 2.59 (t, 2H, J=7.6 Hz), 1.88-1.78 (m, 2H), 1.61-1.60 (m, 2H), 1.43-1.23 (m, 6H), 0.87 (s, 9H), 0.07 (s, 3H), −0.03 (s, 3H); ¹³C NMR (150 MHz, CDCl₃) δ 165.2, 142.9, 133.6, 129.9, 128.5 (2C), 128.4 (2C), 125.7, 68.6, 36.4, 36.0, 31.5, 29.2, 25.8 (3C), 25.1, 18.3, −4.9, −5.0; ESI-TOF m/z 408.2118 (M+H⁺, C₂₂H₃₄ClNO₂Si requires 408.2120).

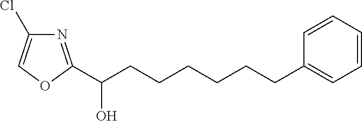

1-(4-Chlorooxazol-2-yl)-7-phenylheptan-1-ol (6c). Compound 6c was prepared from 3c (14 mg) following general procedure A. Flash chromatography (40% EtOAc/hexanes) yielded 6c as a colorless oil (9 mg, 88%): ¹H NMR (600 MHz, CDCl₃) δ 7.57 (s, 1H), 7.29-7.26 (m, 2H), 7.19-7.16 (m, 3H), 4.77-4.74 (m, 1H), 2.60 (t, 2H, J=7.7 Hz), 2.53 (d, 1H, J=6.0 Hz), 1.95-1.83 (m, 2H), 1.64-1.59 (m, 2H), 1.45-1.35 (m, 5H); ¹³C NMR (150 MHz, CDCl₃) δ 165.5, 142.8, 134.0, 130.2, 128.5 (2C), 128.4 (2C), 125.7, 67.9, 36.0, 35.5, 31.5, 29.2 (2C), 24.9; ESI-TOF m/z 294.1255 (M+H⁺, C₁₆H₂₀ClNO₂ requires 294.1255).

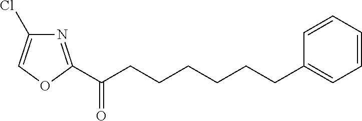

1-(4-Chlorooxazol-2-yl)-7-phenylheptan-1-one (4c). Compound 4c was prepared from 6c (9 mg) following general procedure B. Flash chromatography (20% EtOAc/hexanes) yielded 4c as a colorless oil (8 mg, 93%): ¹H NMR (600 MHz, CDCl₃) δ 7.76 (s, 1H), 7.29-7.26 (m, 2H), 7.19-7.16 (m, 3H), 3.03 (t, 2H, J=7.4 Hz), 2.61 (t, 2H, J=7.7 Hz), 1.77-1.72 (m, 2H), 1.66-1.61 (m, 2H), 1.44-1.35 (m, 4H); ¹³C NMR (150 MHz, CDCl₃) δ 187.7, 156.4, 142.8, 137.2, 131.9, 128.5 (2C), 128.4 (2C), 125.8, 39.2, 36.0, 31.4, 29.1, 29.0, 23.7; ESI-TOF m/z 292.1098 (M+H⁺, C₁₆H₁₈ClNO₂ requires 292.1099).

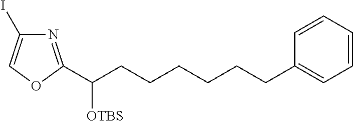

2-(1-(tert-Butyldimethylsilyloxy)-7-phenylheptyl)-4-iodooxazole (3d). Compound 3d was prepared from 3b (100 mg) following general procedure C using iodine (168 mg, 3 equiv) in THF as the electrophile. Preparative TLC (5% EtOAc/hexanes) yielded 3d as a colorless oil (59 mg, 54%): ¹H NMR (600 MHz, CDCl₃) δ 7.62 (s, 1H), 7.29-7.26 (m, 2H), 7.18-7.16 (m, 3H), 4.78 (t, 1H, J=5.9 Hz), 2.59 (t, 2H, J=7.6 Hz), 1.87-1.79 (m, 2H), 1.61-1.59 (m, 2H), 1.41-1.26 (m, 6H), 0.87 (s, 9H), 0.06 (s, 3H), −0.05 (s, 3H); ¹³C NMR (150 MHz, CDCl₃) δ 167.1, 142.9, 142.4, 128.5 (2C), 128.4 (2C), 125.7, 81.5, 68.5, 36.5, 36.0, 31.5, 29.2 (2C), 25.8 (3C), 25.1, 18.3, −4.9, −5.0; ESI-TOF m/z 500.1476 (M+H⁺, C₂₂H₃₄INO₂Si requires 500.1476).

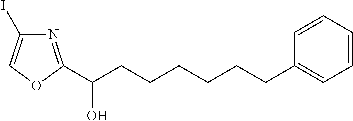

1-(4-Iodooxazol-2-yl)-7-phenylheptan-1-ol (6d). Compound 6d was prepared from 3d (5.7 mg) following general procedure A. Flash chromatography (20-40% EtOAc/hexanes) yielded 6d as a colorless oil (3.4 mg, 77%): ¹H NMR (600 MHz, CDCl₃) δ 7.64 (s, 1H), 7.29-7.26 (m, 2H), 7.18-7.16 (m, 3H), 4.80-4.77 (m, 1H), 2.59 (t, 2H, J=7.7 Hz), 2.45 (d, 1H, J=6.0 Hz), 1.94-1.82 (m, 2H), 1.63-1.60 (m, 2H), 1.44-1.26 (m, 5H); ¹³C NMR (150 MHz, CDCl₃) δ 167.4, 142.9, 142.8, 128.5 (2C), 128.4 (2C), 125.7, 81.6, 67.8, 36.0, 35.6, 31.5, 29.2 (2C), 24.9; ESI-TOF m/z 386.0613 (M+H⁺, C₁₆H₂₀INO₂ requires 386.0611).

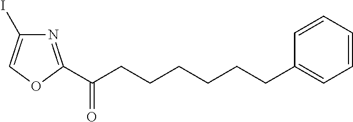

1-(4-Iodooxazol-2-yl)-7-phenylheptan-1-one (4d). Compound 4d was prepared from 6d (3.4 mg) following general procedure B. Flash chromatography (20% EtOAc/hexanes) yielded 4d as a colorless oil (2.9 mg, 85%): ¹H NMR (600 MHz, CDCl₃) δ 7.83 (s, 1H), 7.29-7.26 (m, 2H), 7.18-7.16 (m, 3H), 3.05 (t, 2H, J=7.4 Hz), 2.60 (t, 2H, J=7.7 Hz), 1.76-1.71 (m, 2H), 1.66-1.60 (m, 2H), 1.42-1.37 (m, 4H); ¹³C NMR (150 MHz, CDCl₃) δ 187.6, 158.9, 145.6, 142.8, 128.5 (2C), 128.4 (2C), 125.8, 83.7, 39.2, 36.0, 31.4, 29.1, 29.0, 23.7; ESI-TOF m/z 384.0461 (M+H⁺, C₁₆H₁₈INO₂ requires 384.0455).

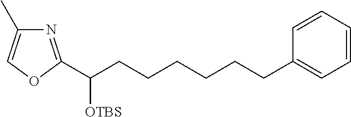

2-(1-(tert-Butyldimethylsilyloxy)-7-phenylheptyl)-4-methyloxazole (3e). Compound 3e was prepared from 3b (24 mg) following general procedure C using methyl iodide (10 μL, 3 equiv) in THF as the electrophile. Preparative TLC (5% EtOAc/hexanes) yielded 3e as a colorless oil (6 mg, 31%): ¹H NMR (600 MHz, CDCl₃) δ 7.30 (s, 1H), 7.28-7.25 (m, 2H), 7.18-7.16 (m, 2H), 4.73 (t, 1H, J=6.0 Hz), 2.58 (t, 2H, J=7.8 Hz), 2.15 (s, 3H), 1.89-1.77 (m, 2H), 1.61-1.57 (m, 2H), 1.43-1.23 (m, 6H), 0.86 (s, 9H), 0.06 (s, 3H), −0.06 (s, 3H); ¹³C NMR (150 MHz, CDCl₃) δ 165.0, 143.0, 136.1, 134.1, 128.5 (2C), 128.4 (2C), 125.7, 68.8, 36.6, 36.1, 31.5, 29.3, 29.2, 25.9, 25.2, 18.4, 11.6, −5.0, −5.1; ESI-TOF m/z 388.2657 (M+H⁺, C₂₃H₃₇NO₂Si requires 388.2666).

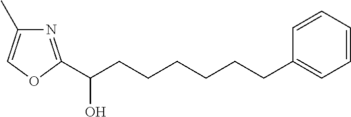

1-(4-Methyloxazol-2-yl)-7-phenylheptan-1-ol (6e). Compound 6e was prepared from 3e (5.7 mg) following general procedure A. Flash chromatography (20-40% EtOAc/hexanes) yielded 6e as a colorless oil (3 mg, 73%): ¹H NMR (600 MHz, CDCl₃) δ 7.32 (s, 1H), 7.28-7.25 (m, 2H), 7.18-7.16 (m, 2H), 4.74-4.72 (m, 1H), 2.61-2.58 (m, 3H), 2.16 (s, 3H), 1.93-1.81 (m, 2H), 1.63-1.59 (m, 3H), 1.44-1.35 (m, 4H); ¹³C NMR (150 MHz, CDCl₃) δ 165.4, 143.0, 136.3, 134.5, 128.5 (2C), 128.4 (2C), 125.7, 68.0, 36.1, 35.7, 31.5, 29.3, 29.2, 25.0, 11.6; ESI-TOF m/z 274.1804 (M+H⁺, C₁₇H₂₃NO₂ requires 274.1801).

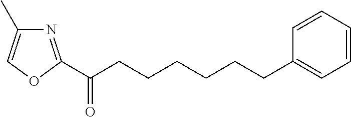

1-(4-Methyloxazol-2-yl)-7-phenylheptan-1-one (4e). Compound 4e was prepared from 6e (2.6 mg) following general procedure B. Flash chromatography (20% EtOAc/hexanes) yielded 4e as a colorless oil (2.5 mg, 97%): ¹H NMR (600 MHz, CDCl₃) δ 7.53 (s, 1H), 7.28-7.25 (m, 2H), 7.18-7.16 (m, 2H), 3.04 (t, 2H, J=7.4 Hz), 2.60 (t, 2H, J=7.7 Hz), 2.27 (s, 3H), 1.77-1.72 (m, 2H), 1.65-1.60 (m, 2H), 1.43-1.37 (m, 4H); ¹³C NMR (150 MHz, CDCl₃) δ 188.7, 157.6, 142.8, 138.8, 137.6, 128.5 (2C), 128.4 (2C), 125.7, 39.2, 36.0, 31.4, 29.2, 29.1, 23.8, 11.7; ESI-TOF m/z 272.1644 (M+H⁺, C₁₇H₂₃NO₂ requires 272.1645).

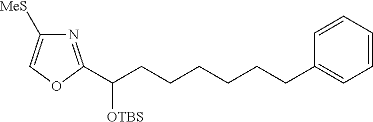

2-(1-(tert-Butyldimethylsilyloxy)-7-phenylheptyl)-4-(methylthio)oxazole (31). Compound 3f was prepared from 3b (33.2 mg) following general procedure C using methyl disulfide (19.8 μL, 3 equiv) as the electrophile. Preparative TLC (7% EtOAc/hexanes) yielded 3f as a colorless oil (12.7 mg, 41%): ¹H NMR (600 MHz, CDCl₃) δ 7.48 (s, 1H), 7.28-7.26 (m, 2H), 7.18-7.16 (m, 2H), 4.76 (t, 1H, J=5.8 Hz), 2.59 (t, 2H, J=7.7 Hz), 2.41 (s, 3H), 1.90-1.78 (m, 2H), 1.62-1.57 (m, 2H), 1.44-1.24 (m, 6H), 0.86 (s, 9H), 0.06 (s, 3H), −0.04 (s, 3H); ¹³C NMR (150 MHz, CDCl₃) δ 166.0, 142.9, 136.6, 135.5, 128.5 (2C), 128.4 (2C), 125.7, 68.8, 36.4, 36.0, 31.5, 29.3, 29.2, 25.8, 25.2, 18.3, 17.0, −5.0, −5.1; ESI-TOF m/z 420.2388 (M+H⁺, C₂₃H₃₇NO₂SSi requires 420.2387).

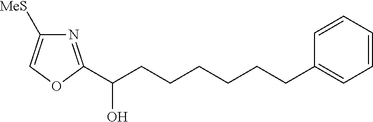

1-(4-(Methylthio)oxazol-2-yl)-7-phenylheptan-1-ol (6f). Compound 6f was prepared from 3f (11.8 mg) following general procedure A. Flash chromatography (40% EtOAc/hexanes) yielded 6f as a colorless oil (7.7 mg, 90%): ¹H NMR (600 MHz, CDCl₃) δ 7.49 (s, 1H), 7.28-7.26 (m, 2H), 7.18-7.16 (m, 2H), 4.78-4.74 (m, 1H), 2.60 (t, 2H, J=7.7 Hz), 2.42 (s, 3H), 1.95-1.82 (m, 2H), 1.63-1.58 (m, 2H), 1.44-1.34 (m, 6H); ¹³C NMR (150 MHz, CDCl₃) δ 166.4, 142.9, 136.9, 135.8, 128.5 (2C), 128.4 (2C), 125.7, 68.0, 36.0, 35.6, 31.5, 29.3, 29.2, 24.9, 16.9; ESI-TOF m/z 306.1515 (M+H⁺, C₁₇H₂₃NO₂S requires 306.1522).

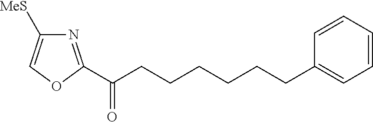

1-(4-(Methylthio)oxazol-2-yl)-7-phenylheptan-1-one (4f). Compound 4f was prepared from 6f (6.9 mg) following general procedure B. Flash chromatography (20% EtOAc/hexanes) yielded 4f as a colorless oil (5.8 mg, 85%): ¹H NMR (600 MHz, CDCl₃) δ 7.65 (s, 1H), 7.28-7.26 (m, 2H), 7.18-7.16 (m, 2H), 3.05 (t, 2H, J=7.4 Hz), 2.61 (t, 2H, J=7.7 Hz), 2.49 (s, 3H), 1.77-1.72 (m, 2H), 1.66-1.61 (m, 2H), 1.43-1.37 (m, 4H); ¹³C NMR (150 MHz, CDCl₃) δ 188.3, 158.0, 142.8, 139.0, 138.7, 128.5 (2C), 128.4 (2C), 125.8, 39.2, 36.0, 31.4, 29.1, 29.1, 23.8, 16.6; ESI-TOF m/z 304.1355 (M+H⁺, C₁₇H₂₁NO₂S requires 304.1366).

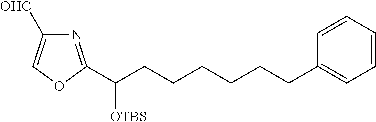

2-(1-(tert-Butyldimethylsilyloxy)-7-phenylheptyl)oxazole-4-carboxaldehyde (3g). Compound 3g was prepared from 3b (35.5 mg) following general procedure D using N,N-dimethylformamide (18 μL, 3 equiv). Preparative TLC (7.5% EtOAc/hexanes) provided 3g as a colorless oil (11.9 mg, 38%): ¹H NMR (600 MHz, CDCl₃) δ 9.94 (s, 1H), 8.22 (s, 1H), 7.28-7.26 (m, 2H), 7.18-7.16 (m, 3H), 4.84 (t, 1H, J=6.0 Hz), 2.59 (t, 2H, J=7.7 Hz), 1.91-1.81 (m, 2H), 1.62-1.58 (m, 2H), 1.42-1.26 (m, 6H), 0.87 (s, 9H), 0.08 (s, 3H), −0.03 (s, 3H); ¹³C NMR (150 MHz, CDCl₃) δ 184.4, 166.8, 144.3, 142.8, 140.6, 128.5 (2C), 128.4 (2C), 125.7, 68.6, 36.4, 36.0, 31.5, 29.2, 25.8, 25.1, 18.3, −4.99, −5.0; ESI-TOF m/z 402.2457 (M+H⁺, C₂₃H₃₅NO₃Si requires 402.2458).

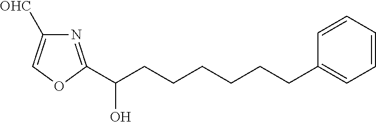

2-(1-Hydroxy-7-phenylheptyl)oxazole-4-carboxaldehyde (6g). Compound 6g was prepared from 3g (11.4 mg) following general procedure A. Flash chromatography (40-60% EtOAc/hexanes) yielded 6g as a colorless oil (6.0 mg, 73%): ¹HNMR (600 MHz, CDCl₃) δ 9.94 (s, 1H), 8.24 (s, 1H), 7.28-7.26 (m, 2H), 7.19-7.16 (m, 3H), 4.86-4.84 (m, 1H), 2.60 (t, 3H, J=7.7 Hz), 1.99-1.86 (m, 2H), 1.64-1.59 (m, 2H), 1.45-1.35 (m, 5H); ¹³C NMR (150 MHz, CDCl₃) δ 183.9, 167.2, 144.8, 142.8, 140.6, 128.5 (2C), 128.4 (2C), 125.8, 67.8, 36.0, 35.6, 31.5, 29.2, 29.2, 24.9; ESI-TOF m/z 288.1592 (M+H⁺, C₁₇H₂₁NO₃ requires 288.1594).

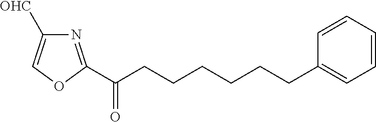

2-(7-Phenylheptanoyl)oxazole-4-carbaldehyde (4g). Compound 4g was prepared from 6g (5.7 mg) following general procedure B. Flash chromatography (30% EtOAc/hexanes) yielded 4g as a colorless oil (4.7 mg, 82%): ¹H NMR (600 MHz, CDCl₃) δ 10.01 (s, 1H), 8.38 (s, 1H), 7.29-7.26 (m, 2H), 7.19-7.16 (m, 3H), 3.12 (t, 2H, J=7.4 Hz), 2.61 (t, 3H, J=7.7 Hz), 1.80-1.75 (m, 2H), 1.66-1.61 (m, 2H), 1.46-1.36 (m, 4H); ¹³C NMR (150 MHz, CDCl₃) δ 188.3, 184.0, 158.0, 145.5, 142.7, 141.4, 128.5 (2C), 128.4 (2C), 125.8, 39.5, 36.0, 31.4, 29.1, 29.0, 23.6; ESI-TOF m/z 286.1438 (M+H⁺, C₁₇H₁₉NO₃ requires 286.1438).

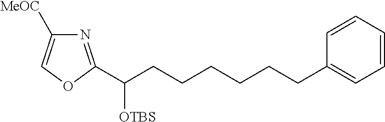

1-(2-(1-(tert-Butyldimethylsilyloxy)-7-phenylheptyl)oxazol-4-yl)ethanone (3h). Compound 3h was prepared from 3b (28.6 mg) following general procedure D using N,N-dimethylacetamide (18 μL, 3 equiv). Preparative TLC (5% EtOAc/hexanes) provided 3h as a colorless oil (4.5 mg, 17%): ¹H NMR (600 MHz, CDCl₃) δ 8.15 (s, 1H), 7.29-7.25 (m, 2H), 7.19-7.15 (m, 3H), 4.82 (t, 1H, J=6.0 Hz), 2.59 (t, 2H, J=7.7 Hz), 2.52 (s, 3H), 1.90-1.78 (m, 2H), 1.63-1.56 (m, 2H), 1.42-1.26 (m, 6H), 0.87 (s, 9H), 0.08 (s, 3H), −0.04 (s, 3H); ¹³C NMR (150 MHz, CDCl₃) δ 193.0, 165.9, 142.9, 142.2, 140.7, 128.5 (2C), 128.4 (2C), 125.7, 68.6, 36.5, 36.0, 31.5, 29.2, 27.6, 25.8, 25.1, 18.3, −4.9, −5.0; ESI-TOF m/z 416.2632 (M+H⁺, C₂₄H₃₇NO₃Si requires 416.2615).

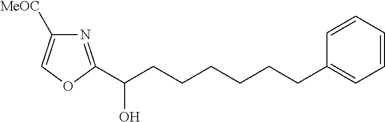

1-(2-(1-Hydroxy-7-phenylheptyl)oxazol-4-yl)ethanone (6h). Compound 6h was prepared from 3h (4.5 mg) following general procedure A. Flash chromatography (40-50% EtOAc/hexanes) yielded 6h as a colorless oil (2.3 mg, 70%): ¹H NMR (600 MHz, CDCl₃) δ 8.17 (s, 1H), 7.29-7.26 (m, 2H), 7.19-7.16 (m, 3H), 4.84-4.81 (m, 1H), 2.60 (t, 2H, J=7.7 Hz), 2.53 (s, 3H), 2.47 (d, 1H, J=5.9 Hz), 1.98-1.85 (m, 2H), 1.64-1.59 (m, 2H), 1.46-1.35 (m, 5H); ¹³C NMR (150 MHz, CDCl₃) δ 192.6, 166.2, 142.8, 142.6, 140.7, 128.5 (2C), 128.4 (2C), 125.8, 67.9, 36.0, 35.6, 31.5, 29.2, 29.2, 27.6, 24.9; ESI-TOF m/z 302.1751 (M+H⁺, C₁₈H₂₃NO₃ requires 302.1751).

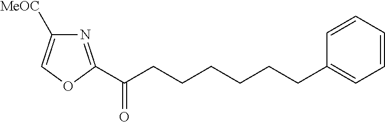

1-(4-Acetyloxazol-2-yl)-7-phenylheptan-1-one (4h). Compound 4h was prepared from 6h (2.1 mg) following general procedure B. Flash chromatography (30% EtOAc/hexanes) yielded 4h as a colorless oil (1.8 mg, 86%): ¹H NMR (600 MHz, CDCl₃) δ 8.31 (s, 1H), 7.29-7.26 (m, 2H), 7.19-7.16 (m, 3H), 3.10 (t, 2H, J=7.4 Hz), 2.61 (t, 2H, J=7.7 Hz), 2.60 (s, 3H), 1.79-1.74 (m, 2H), 1.67-1.62 (m, 2H), 1.46-1.38 (m, 4H); ¹³C NMR (150 MHz, CDCl₃) δ 192.5, 188.5, 157.3, 144.1, 142.8, 141.8, 128.5 (2C), 128.4 (2C), 125.8, 39.4, 36.0, 31.4, 29.1, 29.0, 27.6, 23.7; ESI-TOF m/z 302.1751 (M+H⁺, C₁₈H₂₃NO₃ requires 302.1751).

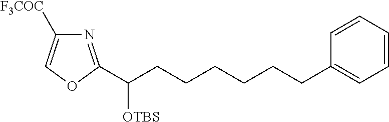

1-(2-(1-(tert-Butyldimethylsilyloxy)-7-phenylheptyl)oxazol-4-yl)-2,2,2-trifluoroethanone (3i). Compound 3i was prepared from 3b (30.9 mg) following general procedure D using trifluoroethyl acetate (24 μL, 3 equiv) as the electrophile. Flash chromatography (10% EtOAc/hexanes) yielded 3i as a clear oil (13.0 mg, 41%): ¹H NMR (600 MHz, CDCl₃) δ 8.41 (s, 1H), 7.28-7.26 (m, 2H), 7.19-7.16 (m, 3H), 4.90-4.87 (m, 1H), 2.61-2.58 (m, 2H), 1.94-1.81 (m, 2H), 1.62-1.57 (m, 2H), 1.40-1.23 (m, 6H), 0.88 (s, 9H), 0.09 (s, 3H), 0.01 (s, 3H); ¹³C NMR (150 MHz, CDCl₃) δ 174.0 (q, J=40 Hz), 170.2, 147.4, 142.8, 134.2, 128.5 (2C), 128.4 (2C), 125.8, 117.0 (q, J=284 Hz), 68.7, 36.4, 36.0, 31.5, 29.9, 29.2, 25.8, 25.0, 18.3, −5.0, −5.1; ESI-TOF m/z 470.2348 (M+H⁺, C₂₄H₃₄F₃NO₃Si requires 470.2333).

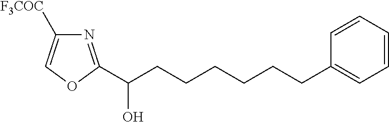

2,2,2-Trifluoro-1-(2-(1-hydroxy-7-phenylheptyl)oxazol-4-yl)ethanone (6i). Compound 3i (13 mg) was dissolved in THF (0.28 mL). Two drops of pyridine were added and the solution was chilled to 0° C. After stirring for 10 min, a drop of HF-pyridine was added. The solution was shaken for 3 days before being quenched with addition of a 1:1 mixture of saturated aqueous NaHCO₃ and EtOAc. The organic layer was washed with aqueous 1 N HCl (2×) and saturated aqueous NaCl and dried over Na₂SO₄. In vacuo evaporation yielded the crude product which was purified by column chromatography (50% EtOAc/hexanes) to give 6i as a clear oil (3.0 mg, 31%): ¹H NMR (600 MHz, CDCl₃) δ 8.42 (s, 1H), 7.29-7.26 (m, 2H), 7.19-7.16 (m, 3H), 4.88 (t, 1H, J=5.4 Hz), 2.61 (t, 2H, J=7.8 Hz), 1.98-1.92 (m, 2H), 1.64-1.59 (m, 2H), 1.46-1.35 (m, 6H); ¹³C NMR (150 MHz, CDCl₃) δ 173.8 (q, J=37.8 Hz), 167.6, 147.5, 142.8, 134.3, 128.5 (2C), 128.4 (2C), 125.8, 116.9 (q, J=288 Hz), 67.8, 36.0, 35.5, 31.4, 29.2 (2C), 24.9; ESI-TOF m/z 356.1480 (M+H⁺, C₁₈H₂₀F₃NO₃ requires 356.1468).

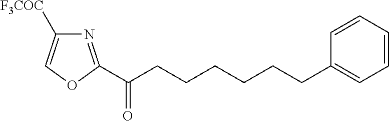

7-Phenyl-1-(4-(2,2,2-trifluoroacetypoxazol-2-yl)heptan-1-one (4i). Compound 4i was prepared from 6i (3.0 mg) following general procedure B. Flash chromatography (40% EtOAc/hexanes) yielded 4i (3.0 mg, 100%) as a clear oil: ¹H NMR (600 MHz, CDCl₃) δ 8.56 (s, 1H), 7.29-7.26 (m, 2H), 7.19-7.16 (m, 3H), 3.15 (t, 2H, J=7.8 Hz), 2.62 (t, 2H, J=7.2 Hz), 1.80-1.74 (m, 2H), 1.66-1.63 (m, 2H), 1.45-1.37 (m, 4H); ¹³C NMR (150 MHz, CDCl₃) δ 188.0, 173.9 (q, J=38.1 Hz), 158.0, 148.8, 142.7, 135.3, 128.5 (2C), 128.4 (2C), 125.8, 116.8 (q, J=287 Hz), 39.6, 36.0, 31.4, 29.0, 28.9, 23.5; ESI-TOF m/z 354.1320 (M+H⁺, C₁₈H₁₈F₃NO₃ requires 354.1312).

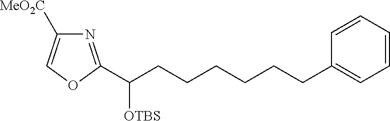

Methyl 2-(1-(tert-Butyldimethylsilyloxy)-7-phenylheptyl)oxazole-4-carboxylate (3j). Compound 3j was prepared from 3b (27.1 mg) following general procedure C using Mander's reagent (MeO₂CCN, 24 μL, 5 equiv) as the electrophile. Flash chromatography (10% EtOAc/hexanes) yielded 3j as a colorless oil (9.5 mg, 37%): ¹H NMR (600 MHz, CDCl₃) δ 8.20 (s, 1H), 7.29-7.25 (m, 2H), 7.19-7.15 (m, 3H), 4.84 (t, 1H, J=6.0 Hz), 3.92 (s, 3H), 2.58 (t, 2H, J=7.7 Hz), 1.90-1.78 (m, 2H), 1.63-1.56 (m, 2H), 1.42-1.26 (m, 6H), 0.86 (s, 9H), 0.07 (s, 3H), −0.06 (s, 3H); ¹³C NMR (150 MHz, CDCl₃) δ 166.3, 161.8, 144.1, 142.9, 133.0, 128.5 (2C), 128.4 (2C), 125.7, 68.6, 52.3, 36.6, 36.0, 31.5, 29.2, 29.2, 25.8, 25.1, 18.3, −4.98, −5.0; ESI-TOF m/z 432.2565 (M+H⁺, C₂₄H₃₇NO₄Si requires 432.2564).

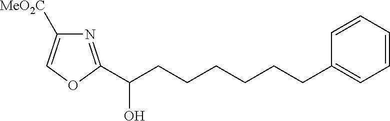

Methyl 2-(1-Hydroxy-7-phenylheptyl)oxazole-4-carboxylate (6j). Compound 6j was prepared from 3j (18.5 mg) following general procedure A. Flash chromatography (40% EtOAc/hexanes) yielded 6j as a colorless oil (11.4 mg, 84%): ¹H NMR (600 MHz, CDCl₃) δ 8.20 (s, 1H), 7.28-7.25 (m, 2H), 7.18-7.15 (m, 3H), 4.84-4.81 (m, 1H), 3.92 (s, 3H), 2.58 (t, 2H, J=7.7 Hz), 2.45 (d, 1H, J=6.1 Hz), 1.97-1.85 (m, 2H), 1.62-1.57 (m, 2H), 1.44-1.32 (m, 5H); ¹³C NMR (150 MHz, CDCl₃) δ 166.6, 161.6, 144.2, 142.8, 133.2, 128.5 (2C), 128.4 (2C), 125.8, 67.9, 52.4, 36.0, 35.6, 31.5, 29.2 (2C), 24.9; ESI-TOF m/z 318.1696 (M+H⁺, C₁₈H₂₃NO₄ requires 318.1700).

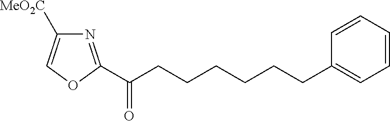

Methyl 2-(7-PhenylheptanoyDoxazole-4-carboxylate (4j). Compound 4j was prepared from 6j (2.4 mg) following general procedure B. Flash chromatography (20% EtOAc/hexanes) yielded 4j as a colorless oil (2.3 mg, 97%): ¹H NMR (600 MHz, CDCl₃) δ 8.36 (s, 1H), 7.28-7.26 (m, 2H), 7.18-7.16 (m, 3H), 3.97 (s, 3H), 3.14 (t, 2H, J=7.4 Hz), 2.58 (t, 2H, J=7.7 Hz), 1.77-1.72 (m, 2H), 1.65-1.60 (m, 2H), 1.44-1.33 (m, 4H); ¹³C NMR (150 MHz, CDCl₃) δ 188.4, 160.9, 157.8, 146.2, 142.8, 134.5, 128.5 (2C), 128.4 (2C), 125.8, 52.7, 39.4, 36.0, 31.4, 29.1, 29.0, 23.5; ESI-TOF m/z 316.1543 (M+H⁺, C₁₈H₂₁NO₄ requires 316.1543).

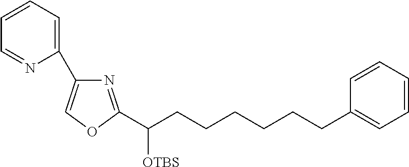

2-(1-(tert-Butyldimethylsilyloxy)-7-phenylheptyl)-4-(pyridin-2-yl)oxazole (3k). Compound 3k was prepared from 3b (51.9 mg) following general procedure E using 2-(tributylstannyl)pyridine (73 μL). Flash chromatography (10% EtOAc/hexanes) yielded 3k as a colorless oil (43 mg, 83%): ¹H NMR (600 MHz, CDCl₃) δ 8.59 (s, 1H), 8.27 (s, 1H), 7.91 (d, 1H, J=7.2 Hz), 7.76 (t, 1H, J=7.8 Hz), 7.27-7.25 (m, 2H), 7.24-7.22 (m, 1H), 7.17-7.15 (m, 3H), 4.85 (t, 1H, J=6.6 Hz), 2.59 (t, 2H, J=7.8 Hz), 1.93-1.85 (m, 2H), 1.68-1.59 (m, 2H), 1.44-1.26 (m, 6H), 0.88 (s, 9H), 0.09 (s, 3H), −0.01 (s, 3H); ¹³C NMR (150 MHz, CDCl₃) δ 165.9, 150.4, 149.3, 142.9, 142.5, 138.6, 128.5 (2C), 128.4, 128.3 (2C), 125.7, 122.7, 120.6, 68.6, 36.6, 36.0, 31.5, 29.3 (2C), 25.9 (3C), 25.2, 18.4, −4.9, −5.0; ESI-TOF m/z 451.2772 (M+H⁺, C₂₇H₃₈N₂O₂Si requires 451.2775).

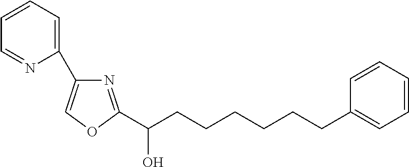

7-Phenyl-1-(4-(pyridin-2-yl)oxazol-2-yl)heptan-1-ol (6k). Compound 6k was prepared from 3k (41.2 mg) following general procedure A. Flash chromatography (40% EtOAc/hexanes) yielded 6k as a colorless oil (22.0 mg, 72%): ¹HNMR (600 MHz, CDCl₃) δ 8.60-8.58 (m, 1H), 8.22 (s, 1H), 7.88 (d, 1H, J=8.4 Hz), 7.76 (dt, 1H, J=1.8, 7.8 Hz), 7.28-7.25 (m, 2H), 7.23-7.21 (m, 1H), 7.18-7.16 (m, 3H), 4.87-4.84 (m, 1H), 2.60 (t, 2H, J=7.8 Hz), 2.01-1.90 (m, 2H), 1.49-1.26 (m, 8H); ¹³C NMR (150 MHz, CDCl₃) δ 166.1, 150.6, 149.7, 142.9, 141.0, 137.0 (2C), 128.5 (2C), 128.4 (2C), 125.7, 123.0, 120.5, 68.0, 36.0, 35.7, 31.5, 29.3, 29.2, 25.0; ESI-TOF m/z 337.1912 (M+H⁺, C₂₁H₂₄N₂O₂ requires 337.1910).

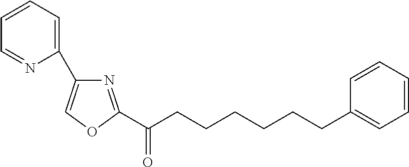

7-Phenyl-1-(4-(pyridin-2-yl)oxazol-2-yl)heptan-1-one (4k). Compound 4k was prepared from 6k (107 mg) following general procedure B. Flash chromatography (20% EtOAc/hexanes) yielded 4k as a white solid (87.8 mg, 83%): ¹HNMR (600 MHz, CDCl₃) δ 8.63-8.61 (m, 1H), 8.40 (s, 1H), 7.99 (d, 1H, J=7.8 Hz), 7.81-7.78 (m, 1H), 7.29-7.26 (m, 3H), 7.23-7.21 (m, 1H), 7.19-7.16 (m, 3H), 3.14 (t, 2H, J=7.8 Hz), 2.62 (t, 2H, J=7.8 Hz), 1.82-1.77 (m, 2H), 1.68-1.63 (m, 2H), 1.48-1.40 (m, 4H); ¹³C NMR (150 MHz, CDCl₃) δ 188.8, 157.9, 149.9, 149.8, 142.9, 142.8, 139.5, 137.1, 128.5 (2C), 128.4 (2C), 125.8, 123.6, 120.6, 39.3, 36.0, 31.4, 29.1 (2C), 23.8; ESI-TOF m/z 335.1753 (M+H⁺, C₂₁H₂₂N₂O₂ requires 335.1754).

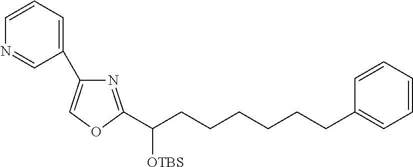

2-(1-(tert-Butyldimethylsilyloxy)-7-phenylheptyl)-4-(pyridin-3-yl)oxazole (31). Compound 3l was prepared from 3b (19.4 mg) following general procedure E using 3-(tributylstannyl)pyridine (13.7 μL, 1 equiv) and toluene. Flash chromatography (10% EtOAc/hexanes) yielded 3l as a colorless oil (14 mg, 74%): ¹H NMR (600 MHz, CDCl₃) δ 8.96 (d, 1H, J=1.8 Hz), 8.56 (dd, 1H, J=1.2, 4.8 Hz), 8.06 (dt, 1H, J=1.8, 7.8 Hz), 7.94 (s, 1H), 7.35-7.33 (m, 1H), 7.28-7.25 (m, 2H), 7.18-7.15 (m, 3H), 4.86-4.84 (m, 1H), 2.59 (t, 2H, J=7.8 Hz), 1.93-1.87 (m, 2H), 1.66-1.58 (m, 2H), 1.39-1.27 (m, 6H), 0.89 (s, 9H), 0.10 (s, 3H), 0.00 (s, 3H); ¹³C NMR (150 MHz, CDCl₃) δ 166.3, 149.1, 147.1, 142.9, 137.8, 134.0, 133.1, 128.5 (2C), 128.4 (2C), 127.4, 125.7, 123.8, 68.8, 36.6, 36.0, 31.5, 29.3, 25.8, 25.2, 18.4, −4.9, −5.0; ESI-TOF m/z 451.2774 (M+H⁺, C₂₇H₃₈N₂O₂Si requires 451.2775).

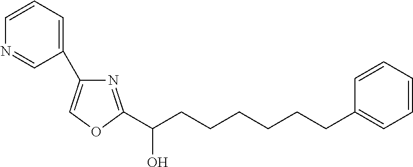

7-Phenyl-1-(4-(pyridin-3-yl)oxazol-2-yl)heptan-1-ol (6l). Compound 6l was prepared from 3l (13.8 mg) following general procedure A. Flash chromatography (50-100% EtOAc/hexanes) yielded 6l as a colorless oil (7.9 mg, 77%): ¹H NMR (600 MHz, CDCl₃) δ 8.99 (d, 1H, J=2.4 Hz), 8.55 (dd, 1H, J=1.8, 4.8 Hz), 8.04 (dt, 1H, J=1.8, 8.4 Hz), 7.94 (s, 1H), 7.35-7.33 (m, 1H), 7.28-7.25 (m, 2H), 7.18-7.16 (m, 3H), 4.87-4.84 (m, 1H), 2.61 (t, 2H, J=7.8 Hz), 2.01-1.89 (m, 2H), 1.64-1.59 (m, 2H), 1.48-1.34 (m, 6H); ¹³C NMR (150 MHz, CDCl₃) δ 166.7, 149.2, 147.1, 142.9, 137.9, 134.5, 133.1, 128.5 (2C), 128.4 (2C), 127.1, 125.7, 123.8, 67.9, 36.0, 35.7, 31.5, 29.3, 29.2, 25.0; ESI-TOF m/z 337.1908 (M+H⁺, C₂₁H₂₄N₂O₂ requires 337.1910).

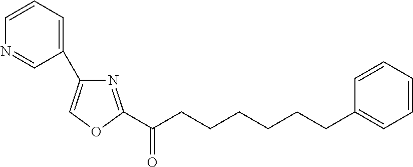

7-Phenyl-1-(4-(pyridin-3-yl)oxazol-2-yl)heptan-1-one (4l). Compound 4l was prepared from 6l (7.4 mg) following general procedure B. Flash chromatography (50% EtOAc/hexanes) yielded 4l as a colorless oil (5.8 mg, 79%): ¹H NMR (600 MHz, CDCl₃) δ 9.03-9.02 (m, 1H), 8.63 (dd, 1H, J=1.8, 4.8 Hz), 8.13 (s, 1H), 8.13 (dt, 1H, J=1.8, 7.8 Hz), 7.41-7.39 (m, 1H), 7.28-7.26 (m, 2H), 7.18-7.16 (m, 3H), 3.15 (t, 2H, J=7.2 Hz), 2.63 (t, 1H, J=7.8 Hz), 1.82-1.77 (m, 2H), 1.68-1.62 (m, 2H), 1.48-1.38 (m, 4H); ¹³C NMR (150 MHz, CDCl₃) δ 188.6, 158.2, 150.0, 147.2, 142.8, 139.9, 136.8, 133.4, 128.5 (2C), 128.4 (2C), 126.3, 125.8, 123.9, 39.4, 36.0, 31.4, 29.1, 23.9; ESI-TOF m/z 335.1761 (M+H⁺, C₂₁H₂₂N₂O₂ requires 335.1754).

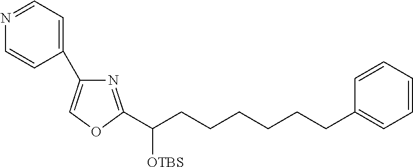

2-(1-(tent-Butyldimethylsilyloxy)-7-phenylheptyl)-4-(pyridin-4-yl)oxazole (3m). Compound 3m was prepared from 3b (17.7 mg) following general procedure E using 4-(tributylstannyl)pyridine (12.5 μL, 1.0 equiv) and toluene. Flash chromatography (10% EtOAc/hexanes) yielded 3m as a colorless oil (14 mg, 82%): ¹H NMR (600 MHz, CDCl₃) δ 8.64 (d, 2H, J=6.0 Hz), 8.02 (s, 1H), 7.62-7.61 (m, 1H), 7.28-7.25 (m, 2H), 7.18-7.15 (m, 3H), 4.86-4.84 (m, 1H), 2.59 (t, 2H, J=7.8 Hz), 1.94-1.85 (m, 2H), 1.67-1.58 (m, 2H), 1.48-1.29 (m, 6H), 0.89 (s, 9H), 0.10 (s, 3H), 0.00 (s, 3H); ¹³C NMR (150 MHz, CDCl₃) δ 166.5, 150.4 (2C), 142.9, 138.8, 138.5, 135.7, 128.5 (2C), 128.4 (2C), 125.7, 120.0 (2C), 68.8, 36.5, 36.0, 31.5, 29.2, 25.8, 25.2, 18.4, −4.9, −5.0; ESI-TOF m/z 451.2769 (M+H⁺, C₂₇H₃₈N₂O₂Si requires 451.2775).

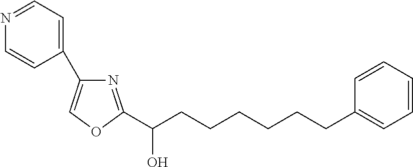

7-Phenyl-1-(4-(pyridin-4-yl)oxazol-2-yl)heptan-1-ol (6m). Compound 6m was prepared from 3m (14.1 mg) following general procedure A. Flash chromatography (50-100% EtOAc/hexanes) yielded 6m as a colorless oil (8.0 mg, 76%): ¹H NMR (600 MHz, CDCl₃) δ 8.63-8.62 (m, 2H), 8.03 (s, 1H), 7.61-7.60 (m, 1H), 7.28-7.25 (m, 2H), 7.18-7.15 (m, 3H), 4.86-4.84 (m, 1H), 2.59 (t, 2H, J=7.8 Hz), 2.00-1.89 (m, 2H), 1.64-1.59 (m, 2H), 1.48-1.33 (m, 6H); ¹³C NMR (150 MHz, CDCl₃) δ 166.7, 150.4 (2C), 142.8, 138.6, 138.4, 136.0, 128.5 (2C), 128.4 (2C), 125.7, 120.0 (2C), 67.9, 36.0, 35.7, 31.5, 29.3, 29.2, 25.0; ESI-TOF m/z 337.1912 (M+H⁺, C₂₁H₂₄N₂O₂ requires 337.1910).

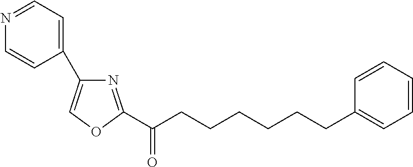

7-Phenyl-1-(4-(pyridin-4-yl)oxazol-2-yl)heptan-1-one (4m). Compound 4m was prepared from 6m (7.3 mg) following general procedure B. Flash chromatography (50% EtOAc/hexanes) yielded 4m as a colorless oil (5.9 mg, 82%): ¹H NMR (600 MHz, CDCl₃) δ 8.71-8.70 (m, 2H), 8.20 (s, 1H), 7.70-7.68 (m, 1H), 7.28-7.26 (m, 2H), 7.18-7.16 (m, 3H), 3.14 (t, 2H, J=7.2 Hz), 2.62 (t, 2H, J=7.2 Hz), 1.82-1.77 (m, 2H), 1.68-1.63 (m, 2H), 1.48-1.38 (m, 4H); ¹³C NMR (150 MHz, CDCl₃) δ 188.6, 158.2, 150.6 (2C), 142.8, 140.4, 138.3, 137.6, 128.5 (2C), 128.4 (2C), 125.8, 120.2 (2C), 39.4, 36.0, 31.4, 29.1, 23.8; ESI-TOF m/z 335.1758 (M+H⁺, C₂₁H₂₂N₂O₂ requires 335.1754).

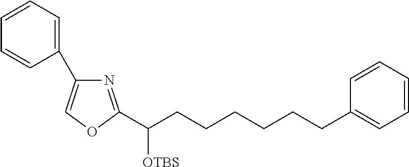

2-(1-(tert-Butyldimethylsilyloxy)-7-phenylheptyl)-4-phenyloxazole (3n). Compound 3n was prepared from 3b (12.2 mg) following general procedure E using tributylphenylstannane (10 μL, 1 equiv). Column chromatography (3% EtOAc/hexanes) yielded 3n as a colorless oil (8.9 mg, 75%): ¹H NMR (600 MHz, CDCl₃) δ 7.86 (s, 1H), 7.74 (d, 2H, J=8.4 Hz), 7.40 (t, 2H, J=7.8 Hz), 7.33-7.30 (m, 1H), 7.28-7.25 (m, 2H), 7.18-7.16 (m, 3H), 4.86 (t, 1H, J=7.2 Hz), 2.59 (t, 2H, J=7.8 Hz), 1.95-1.85 (m, 2H), 1.63-1.58 (m, 2H), 1.39-1.26 (m, 6H), 0.89 (s, 9H), 0.09 (s, 3H), −0.01 (s, 3H); ¹³C NMR (150 MHz, CDCl₃) δ 165.8, 142.9, 140.5, 133.4, 131.2, 128.8 (2C), 128.5 (2C), 128.4 (2C), 128.1 (2C), 125.7 (2C), 68.9, 36.6, 36.0, 31.5 (2C), 29.2, 25.9 (3C), 25.3, 18.4, −4.9, −5.0; ESI-TOF m/z 450.2822 (M+H⁺, C₂₈H₃₉NO₂Si requires 450.2823).

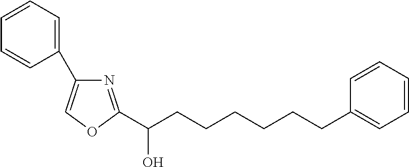

7-Phenyl-1-(4-phenyloxazol-2-yl)heptan-1-ol (6n). Compound 6n was prepared from 3n (8.9 mg) following general procedure A. Flash chromatography (30% EtOAc/hexanes) yielded 6n as a white solid (5.7 mg, 86%): ¹H NMR (600 MHz, CDCl₃) δ 7.88 (s, 1H), 7.74 (d, 2H, J=7.2 Hz), 7.41 (t, 2H, J=7.8 Hz), 7.34-7.31 (m, 1H), 7.28-7.25 (m, 2H), 7.18-7.16 (m, 3H), 4.85-4.83 (m, 1H), 2.60 (t, 2H, J=7.8 Hz), 2.00-1.87 (m, 2H), 1.64-1.59 (m, 2H), 1.51-1.34 (m, 6H); ¹³C NMR (150 MHz, CDCl₃) δ 166.1, 142.9, 140.7, 133.8, 130.8, 128.9 (2C), 128.5 (2C), 128.4 (2C), 128.3 (2C), 125.7 (2C), 68.0, 36.0, 35.7, 31.5, 29.3, 29.2, 25.0; ESI-TOF m/z 336.1956 (M+H⁺, C₂₂H₂₅NO₂ requires 336.1958).

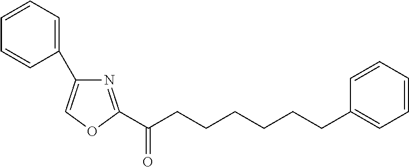

7-Phenyl-1-(4-phenyloxazol-2-yl)heptan-1-one (4n). Compound 4n was prepared from 6n (5.7 mg) following general procedure B. Flash chromatography (20% EtOAc/hexanes) yielded 4n as a white solid (5.0 mg, 88%): ¹H NMR (600 MHz, CDCl₃) δ 8.05 (s, 1H), 7.81 (d, 2H, J=7.2 Hz), 7.46 (t, 2H, J=7.2 Hz), 7.40-7.37 (m, 1H), 7.28-7.26 (m, 2H), 7.19-7.16 (m, 3H), 3.14 (t, 2H, J=7.2 Hz), 2.62 (t, 2H, J=7.8 Hz), 1.82-1.77 (m, 2H), 1.68-1.63 (m, 2H), 1.48-1.39 (m, 4H); ¹³C NMR (150 MHz, CDCl₃) δ 188.9, 158.0, 142.8 (2C), 136.3, 130.0, 129.1, 129.0 (2C), 128.5 (2C), 128.4 (2C), 126.0 (2C), 125.8, 39.3, 36.0, 31.4, 29.1 (2C), 23.9; ESI-TOF m/z 334.1804 (M+H⁺, C₂₂H₂₅NO₂ requires 334.1801).

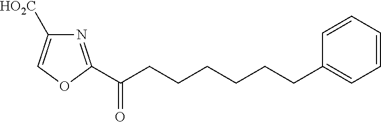

2-(7-Phenylheptanoyl)oxazole-4-carboxylic Acid (4o). Compound 4j (10.6 mg, 1 equiv) was dissolved in a mixture of 3:2 THF/H₂O (0.4 mL) and LiOH (4.2 mg, 3 equiv) was added. The reaction mixture was stirred for 2 h at room temperature before the mixture was acidified using aqueous 1 N HCl. The solution was diluted with EtOAc and the organic layer was separated from the aqueous layer. The aqueous layer was extracted with EtOAc (3×). The combined organic extracts were washed with saturated aqueous NaCl and dried over Na₂SO₄. Evaporation in vacuo yielded the crude acid which was purified by flash chromatography (60-80% EtOAc/hexanes) to provide compound 4o as a white solid (6.6 mg, 65%): ¹HNMR (600 MHz, CD₃OD) δ 8.67 (s, 1H), 7.24-7.21 (m, 2H), 7.16-7.11 (m, 3H), 3.08 (t, 2H, J=7.3 Hz), 2.61 (t, 2H, J=7.6 Hz), 1.74-1.69 (m, 2H), 1.66-1.61 (m, 2H), 1.44-1.36 (m, 4H); ¹³C NMR (150 MHz, CD₃OD) δ 189.3, 163.3, 159.2, 148.1, 143.9, 135.9, 129.4 (2C), 129.2 (2C), 126.6, 39.4, 36.8, 32.5, 30.0, 29.9, 24.5; ESI-TOF m/z 302.1388 (M+H⁺, C₁₇H₁₉NO₄ requires 302.1387).

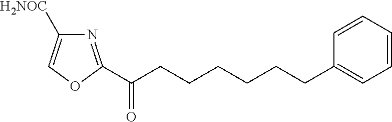

2-(7-Phenylheptanoyl)oxazole-4-carboxamide (4p). Compound 4j (16.6 mg) was dissolved in methanolic ammonia (1 mL) and the mixture was stirred for 2 h at room temperature. Evaporation in vacuo yielded the crude carboxamide which was purified by flash chromatography (60-80% EtOAc/hexanes) to provide 4p as a white solid (4.5 mg, 28%): ¹H NMR (600 MHz, CDCl₃) δ 8.35 (s, 1H), 7.29-7.26 (m, 2H), 7.19-7.17 (m, 3H), 6.83 (br s), 5.74 (br s), 3.05 (t, 2H, J=7.4 Hz), 2.62 (t, 2H, J=7.7 Hz), 1.79-1.74 (m, 2H), 1.67-1.62 (m, 2H), 1.46-1.37 (m, 4H); ¹³C NMR (150 MHz, CDCl₃) δ 188.1, 161.5, 157.0, 144.1, 142.7, 137.1, 128.5 (2C), 128.4 (2C), 125.8, 39.4, 36.0, 31.4, 29.0 (2C), 23.6; ESI-TOF m/z 301.1554 (M+H⁺, C₁₇H₂₀N₂O₃ requires 301.1547).

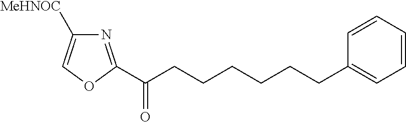

N-Methyl-2-(7-phenylheptanoyl)oxazole-4-carboxamide (4q). Compound 4q was prepared from 2-(7-phenylheptanoyl)oxazole-4-carboxylic acid (4o, 8.4 mg) and methylamine hydrochloride (3.0 mg, 1.5 equiv) following general procedure F. Flash chromatography (50% EtOAc/hexanes) yielded 4q (2.0 mg, 23%) as a white solid: ¹H NMR (600 MHz, CDCl₃) δ 8.31 (s, 1H), 7.29-7.26 (m, 2H), 7.19-7.16 (m, 3H), 6.94 (br s, 1H), 3.05 (t, 2H, J=7.2 Hz), 3.02 (d, 3H, J=5.4 Hz), 2.63 (t, 2H, J=7.8 Hz), 1.78-1.73 (m, 2H), 1.67-1.62 (m, 2H), 1.44-1.37 (m, 4H); ¹³C NMR (150 MHz, CDCl₃) δ 188.1, 160.3, 156.9, 143.2, 142.7, 137.7, 128.5 (2C), 128.4 (2C), 125.8, 39.4, 36.0, 31.4, 29.1, 26.0, 23.4; ESI-TOF m/z 315.1699 (M+H⁺, C₁₈H₂₂N₂O₃ requires 315.1703).

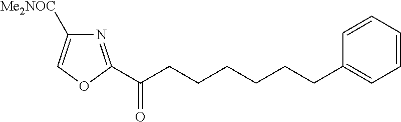

N,N-Dimethyl-2-(7-phenylheptanoyl)oxazole-4-carboxamide (4r). Compound 4r was prepared from 2-(7-phenylheptanoyl)oxazole-4-carboxylic acid (4o, 8.0 mg) and dimethylamine hydrochloride (3.2 mg, 1.5 equiv) following general procedure F. Flash chromatography (50% EtOAc/hexanes) yielded 4r (3.3 mg, 38%) as a white solid: ¹H NMR (600 MHz, CDCl₃) δ 8.28 (s, 1H), 7.29-7.26 (m, 2H), 7.19-7.16 (m, 3H), 3.41 (s, 3H), 3.12 (s, 3H), 3.06 (t, 2H, J=7.2 Hz), 2.61 (t, 2H, J=7.8 Hz), 1.78-1.73 (m, 2H), 1.66-1.61 (m, 2H), 1.44-1.37 (m, 4H); ¹³C NMR (150 MHz, CDCl₃) δ 188.5, 161.2, 156.3, 145.4, 142.7, 138.5, 128.5 (2C), 128.4 (2C), 125.8, 39.3, 38.6, 36.5, 36.0, 31.4, 29.1, 23.8; ESI-TOF m/z 329.1867 (M+H⁺, C₁₉H₂₄N₂O₃ requires 329.1860).

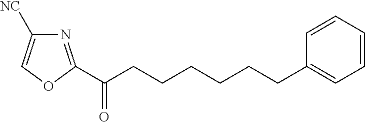

2-(7-Phenylheptanoyl)oxazole-4-carbonitrile (4s). Compound 4p (9 mg, 0.030 mmol) was dissolved in 1,4-dioxane (760 μL) and pyridine (6 μL, 2.5 equiv) and trifluoroacetic anhydride (5.3 μL, 1.3 equiv) were added. The reaction mixture stirred for 2 h at room temperature. The mixture was diluted with CH₂Cl₂ and the combined organic layers were washed with saturated aqueous NaCl and dried over Na₂SO₄. Evaporation in vacuo yielded the crude nitrile which was purified by flash chromatography (20% EtOAc/hexanes) to afford 4s as a white solid (5.9 mg, 70%): ¹H NMR (600 MHz, CDCl₃) δ 8.29 (s, 1H), 7.29-7.26 (m, 2H), 7.19-7.17 (m, 3H), 3.07 (t, 2H, J=7.2 Hz), 2.61 (t, 2H, J=7.8 Hz), 1.78-1.73 (m, 2H), 1.66-1.61 (m, 2H), 1.45-1.36 (m, 4H); ¹³C NMR (150 MHz, CDCl₃) δ 187.4, 157.8, 148.1, 142.7, 128.5 (2C), 128.4 (2C), 125.8, 116.6, 110.7, 39.5, 36.0, 31.3, 29.0 (2C), 23.6; ESI-TOF m/z 283.1450 (M+H⁺, C₁₇H₁₈N₂O₂ requires 283.1441).

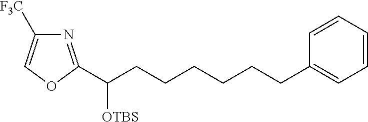

2-(1-(tert-Butyldimethylsilyloxy)-7-phenylheptyl)-4-(trifluoromethyl)oxazole (3t). 2-(1-(tert-Butyldimethylsilyloxy)-7-phenylheptyl)-4-iodooxazole (3d, 59 mg), HMPA (103 μL, 5 equiv), CuI (27 mg, 1.2 equiv), and FSO₂CF₂CO₂CH₃ (75 μL, 5 equiv) were dissolved in DMF (2.36 mL) and the mixture was heated at 70° C. in a sealed vial for 8 h. The mixture was cooled to room temperature, saturated aqueous NH₄Cl was added and the aqueous layer was extracted with ether. The ether layer was washed with saturated aqueous NaHCO₃, washed with saturated aqueous NaCl and dried over Na₂SO₄. Preparative TLC (5% EtOAc/hexanes) yielded 3t as a colorless oil (12.9 mg, 25%): NMR (600 MHz, CDCl₃) δ 7.93 (s, 1H), 7.29-7.26 (m, 2H), 7.19-7.16 (m, 3H), 4.81 (t, 1H, J=5.9 Hz), 2.59 (t, 2H, J=7.7 Hz), 1.90-1.83 (m, 2H), 1.62-1.58 (m, 2H), 1.44-1.26 (m, 6H), 0.87 (s, 9H), 0.07 (s, 3H), −0.04 (s, 3H); ¹³C NMR (150 MHz, CDCl₃) δ 167.2, 142.9, 138.2 (d, J=17.4 Hz), 132.2 (d, J=157.8 Hz), 128.5 (2C), 128.4 (2C), 125.7, 121.5 (d, J=1062 Hz), 68.5, 36.3, 36.0, 31.5, 29.2 (2C), 25.8 (3C), 25.1, 18.3, −5.0, −5.1; ESI-TOF m/z 442.2389 (M+H⁺, C₂₂H₃₄F₃NO₂Si requires 442.2384).

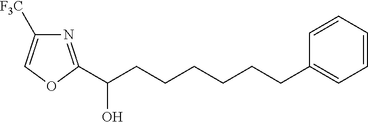

7-Phenyl-1-(4-(trifluoromethyl)oxazol-2-yl)heptan-1-ol (6t). Compound 6t was prepared from 3t (2.2 mg) following general procedure A. Flash chromatography (30% EtOAc/hexanes) yielded 6t as a colorless oil (1.5 mg, 94%): ¹H NMR (600 MHz, CDCl₃) δ 7.96 (s, 1H), 7.29-7.26 (m, 2H), 7.19-7.16 (m, 3H), 4.83 (t, 1H, J=5.8 Hz), 2.60 (t, 2H, J=7.7 Hz), 1.98-1.86 (m, 2H), 1.64-1.59 (m, 2H), 1.47-1.35 (m, 6H); ¹³C NMR (150 MHz, CDCl₃) δ 167.4, 142.8, 138.6 (d, J=17.4 Hz), 132.6 (q, J=159.6 Hz), 128.5 (2C), 128.4 (2C), 125.8, 123.1 (q, J=1063 Hz), 67.8, 36.0, 35.4, 31.5, 29.2 (2C), 24.9; ESI-TOF m/z 328.1512 (M+H⁺, C₁₇H₂₀F₃NO₂ requires 328.1519).

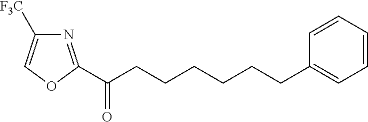

7-Phenyl-1-(4-(trifluoromethyl)oxazol-2-yl)heptan-1-one (4t). Compound 4t was prepared from 6t (8.0 mg) following general procedure B. Flash chromatography (20% EtOAc/hexanes) yielded 4t as a colorless oil (7.6 mg, 95%): ¹H NMR (600 MHz, CDCl₃) δ 8.13 (s, 1H), 7.29-7.26 (m, 2H), 7.19-7.17 (m, 3H), 3.11 (t, 2H, J=7.4 Hz), 2.61 (t, 2H, J=7.7 Hz), 1.78-1.73 (m, 2H), 1.66-1.61 (m, 2H), 1.45-1.36 (m, 4H); ¹³C NMR (150 MHz, CDCl₃) δ 187.9, 158.2, 142.7, 140.7, 134.1 (q, J=162.0 Hz), 128.5 (2C), 128.4 (2C), 125.8, 122.6 (q, J=1066 Hz), 39.4, 36.0, 31.4, 29.0 (2C), 23.6; ESI-TOF m/z 326.1368 (M+H⁺, C₁₇H₁₈F₃NO₂ requires 326.1362).

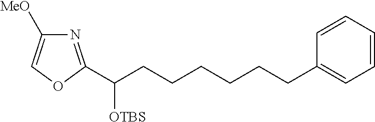

2-(1-(tert-Butyldimethylsilyloxy)-7-phenylheptyl)-4-methoxyoxazole (3u). 2-(1-(tert-Butyldimethylsilyloxy)-7-phenylheptyl)-4-iodooxazole (3d) (5.5 mg, 0.011 mmol) was dissolved in MeOH (0.11 mL) and CuI (1 mg, 0.1 equiv), N,N-dimethylglycine hydrochloride (1 mg, 0.2 equiv), and Cs₂CO₃ (7.2 mg, 2 equiv) were added. The mixture was heated for 8 h in a sealed vial at 110° C. After allowing the reaction mixture to cool to room temperature, it was diluted with EtOAc and washed with water and saturated aqueous NaCl and dried over Na₂SO₄. After concentration in vacuo, column chromatography (5% EtOAc/hexanes) yielded 3u (1.7 mg, 39%) as a clear oil: ¹H NMR (600 MHz, CDCl₃) δ 7.28-7.26 (m, 2H), 7.18-7.16 (m, 3H), 7.05 (s, 1H), 4.67-4.65 (m, 1H), 3.78 (s, 3H), 2.59 (t, 1H, J=7.8 Hz), 1.84-1.78 (m, 2H), 1.61-1.55 (m, 2H), 1.44-1.25 (m, 6H), 0.87 (s, 9H), 0.06 (s, 3H), −0.03 (s, 3H); ¹³C NMR (150 MHz, CDCl₃) δ 162.8, 156.4, 143.0, 128.5 (2C), 128.4 (2C), 125.7, 116.0, 68.9, 57.4, 36.4, 36.1, 31.5, 29.8, 29.3, 25.9, 25.2, 18.4, 14.3, −4.9, −5.0; ESI-TOF m/z 404.2605 (M+H⁺, C₂₃H₃₇NO₃Si requires 404.2615).

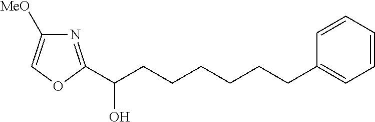

1-(4-Methoxyoxazol-2-yl)-7-phenylheptan-1-ol (6u). Compound 6u was prepared from 3u (5.1 mg) following general procedure A. Flash chromatography (50% EtOAc/hexanes) yielded 6u as a clear oil (3.2 mg, 89%): ¹H NMR (600 MHz, CDCl₃) δ 7.28-7.26 (m, 2H), 7.18-7.16 (m, 3H), 7.08 (s, 1H), 4.68 (t, 1H, J=7.2 Hz), 3.79 (s, 3H), 2.59 (t, 2H, J=7.8 Hz), 1.93-1.79 (m, 2H), 1.63-1.58 (m, 2H), 1.42-1.32 (m, 6H); ¹³C NMR (150 MHz, CDCl₃) δ 163.1, 156.4, 142.9, 128.5 (2C), 128.4 (2C), 125.7, 116.5, 68.1, 57.5, 36.0, 35.6, 31.5, 29.3, 29.2, 24.9; ESI-TOF m/z 290.1750 (M+H⁺, C₁₇H₂₃NO₃ requires 290.1750).

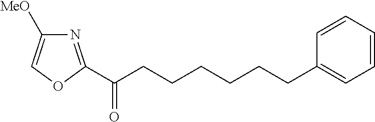

1-(4-Methoxyoxazol-2-yl)-7-phenylheptan-1-one (4u). Compound 4u was prepared from 6u (3.0 mg) following general procedure B. Flash chromatography (25% EtOAc/hexanes) yielded 4u as a yellow oil (2.6 mg, 87%): ¹H NMR (600 MHz, CDCl₃) δ 7.29 (s, 1H), 7.28-7.26 (m, 2H), 7.18-7.16 (m, 3H), 3.90 (s, 3H), 2.99 (t, 2H, J=7.2 Hz), 2.60 (t, 2H, J=7.8 Hz), 1.76-1.70 (m, 2H), 1.65-1.60 (m, 2H), 1.41-1.35 (m, 4H); ¹³C NMR (150 MHz, CDCl₃) δ 188.3, 156.8, 154.0, 142.8, 128.5 (2C), 128.4 (2C), 125.8, 120.7, 57.7, 39.0, 36.0, 31.4, 29.1 (2C), 24.0; ESI-TOF m/z 288.1592 (M+H⁺, C₁₇H₂₁NO₃ requires 288.1594).

Enzyme Assay. ¹⁴C-labeled oleamide was prepared from ¹⁴C-labeled oleic acid as described by Cravatt et al. (Science 1995, 268, 1506). The truncated rat FAAH (rFAAH) was expressed in E. coli and purified as described by Patricelli et al. (Biochemistry 1998, 37, 15177). The purified recombinant rFAAH was used in the inhibition assays unless otherwise indicated. The full-length human FAAH (hFAAH) was expressed in COS-7 cells as described by Giang et al. (Proc. Natl. Acad. Sci. U.S.A. 1997, 94, 2238), and the lysate of hFAAH-transfected COS-7 cells was used in the inhibition assays where explicitly indicated.

The inhibition assays were performed as described by Cravatt et al. (Science 1995, 268, 1506). In brief, the enzyme reaction was initiated by mixing 1 nM of rFAAH (800, 500, or 200 pM rFAAH for inhibitors with Kᵢ≦1-2 nM) with 10 μM of ¹⁴C-labeled oleamide in 500 μL of reaction buffer (125 mM TrisCl, 1 mM EDTA, 0.2% glycerol, 0.02% Triton X-100, 0.4 mM Hepes, pH 9.0) at room temperature in the presence of three different concentrations of inhibitor. The enzyme reaction was terminated by transferring 20 μL of the reaction mixture to 500 μL of 0.1 N HCl at three different time points. The ¹⁴C-labeled oleamide (substrate) and oleic acid (product) were extracted with EtOAc and analyzed by TLC. The Kᵢ of the inhibitor was calculated using a Dixon plot (standard deviations are provided in the Supporting Information tables). Lineweaver-Burk analysis was performed as described confirming competitive, reversible inhibition (Boger et al., J. Med. Chem. 2005, 48, 1849). The selectivity screening was conducted as detailed by Leung et al. (Nature Biotech. 2003, 21, 687).

TABLE 3
% Hydration or Hemiketal Formation^{a}

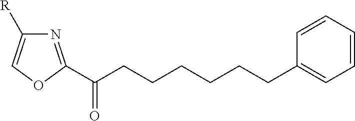

| R | CDCl₃ | CD₃OD |
|---|---|---|
| CHO (4g) | 0% | >95% for CHO |
| COCF₃ (4i) | 33% | >95% for COCF₃ |

^{a}Established by ¹H and ¹³C NMR in the solvent indicated

TABLE 4
Inhibition Results with Calculated Errors

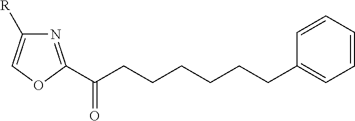

| cmpd | R | Kᵢ, nM |
|---|---|---|
| 4a | H | 48 ± 0.003 |
| 4b | Br | 3.0 ± 0.02 |
| 4c | Cl | 4.0 ± 0.05 |
| 4d | I | 6.5 ± 0.26 |
| 4e | CH₃ | 520 ± 1.0 |
| 4f | SCH₃ | 29 ± 0.2 |
| 4g | CHO | 55 ± 5.0 |
| 4h | COCH₃ | 2.0 ± 0.07 |
| 4i | CF₃CO | 470 ± 2.0 |
| 4j | CO₂CH₃ | 3.4 ± 0.05 |
| 4k | 2-Pyr | 1.9 ± 0.02 |
| 4l | 3-pyr | 18 ± 0.1 |
| 4m | 4-pyr | 1.6 ± 0.01 |
| 4n | Ph | 65 ± 4.4 |
| 4o | CO₂— | 53 ± 1.0 |
| 4p | CONH₂ | 1.6 ± 0.001 |
| 4q | CONHMe | 1.8 ± 0.005 |
| 4r | CONMe₂ | 35 ± 0.1 |
| 4s | CN | 0.5 ± 0.009 |
| 4t | CF₃ | 3.7 ± 0.02 |
| 4u | OMe | 740 ± 0.07 |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A compound of formula I: 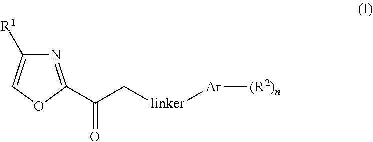
wherein
R¹ is —Y—R^{x};
Y is —CH₂—, —O—, —CH₂O—, —OCH₂—, —C(═O)—, —CO₂—, —OC(═O)—, —S—, —S(O)—, —S(O)₂—, —N(R^{a})—, or a direct bond;
R^{x} is H, halo, (C₁-C₂₀)alkyl, (C₁-C₈)cycloalkyl, trifluoromethyl, aryl, heteroaryl, —CN, —NO₂, or —NR^{a}R^{b};
linker is a (C₁-C₂₀)alkyl chain wherein one to five carbons of the chain are optionally be replaced with O or S, or linker is a direct bond;
Ar is (C₆-C₁₄)aryl;
each R² is independently H, —X—R³, or —X-Ph-X—R³;
n is 1-4;
each X is independently —CH₂—, —O—, —CH₂O—, —OCH₂—, —C(═O)—, —CO₂—, —OC(═O)—, —S—, —S(O)—, —S(O)₂—, —N(R^{a})—, or a direct bond;
each R³ is independently H, (C₁-C₈)alkyl, (C₃-C₈)cycloalkyl, aryl, heteroaryl, —CF₃, —CN, —C(O)(C₁-C₈)alkyl optionally substituted with one, two, or three fluoro substituents, —CO₂(C₁-C₈)alkyl, —CO₂H, —C(O)NR^{a}R^{b}, —OH, —O(C₁-C₈)alkyl, -halo, —NO₂, —NR^{a}R^{b}, —N(R^{a})SO₂R^{b}, —SO₂NR^{a}R^{b}, —S(O)₀₋₂R^{a}, or —CH₂NR^{e}R^{d} wherein R^{c} and R^{d} are each independently H or (C₁-C₈)alkyl, or R^{c} and R^{d} taken together with the nitrogen to which they are attached form a monocyclic saturated heterocyclic group;
each R^{a} and R^{b} are each independently H, (C₁-C₈)alkyl, (C₃-C₈)cycloalkyl, aryl(C₁-C₈)alkyl, or a nitrogen protecting group; and
any alkyl, cycloalkyl, aryl or heteroaryl of R^{x} is optionally substituted with one, two, or three R² groups;
or a pharmaceutically acceptable salt thereof.

2. The compound of claim 1 wherein R¹ is H, halo, (C₁-C₆)alkyl, (C₁-C₆)alkoxy, (C₁-C₆)alkylthio, —CHO, carboxy, (C₁-C₆)alkanoyl, (C₁-C₆)alkoxycarbonyl, trifluoromethyl, trifluoromethoxy, phenyl, pyridyl, —CN, or —C(═O)—NR^{a}R^{b}.

3. The compound of claim 2 wherein R¹ is fluoro, chloro, iodo, methyl, ethyl, propyl, —OMe, —OEt, —SMe, —SEt, —C(═O)Me, —CO₂Me, —CONH₂, —CONH(Me), or —CON(Me)₂.

4. The compound of claim 1 wherein linker is a (C₁-C₈)alkyl or a direct bond.

5. The compound of claim 1 wherein R² is H and n is 1.

6. The compound of claim 1 wherein R₂ is —X—R³; X is —O—, —S—, or a direct bond; and R³ is phenyl.

7. The compound of claim 1 wherein the compound is a compound of formula V:

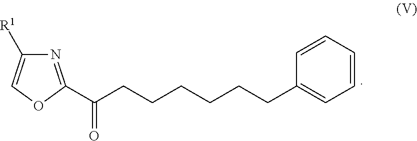

8. The compound of claim 7 wherein R¹ is H, halo, (C₁-C₆)alkyl, (C₁-C₆)alkoxy, (C₁-C₆)alkylthio, —CHO, carboxy, (C₁-C₆)alkanoyl, (C₁-C₆)alkoxycarbonyl, trifluoromethyl, trifluoromethoxy, phenyl, pyridyl, —CN, or —C(═O)—NR^{a}R^{b}.

9. The compound of claim 8 wherein R¹ is fluoro, chloro, iodo, methyl, ethyl, propyl, —OMe, —OEt, —SMe, —SEt, —C(═O)Me, —CO₂Me, —CONH₂, —CONH(Me), or —CON(Me)₂.

10. A compound selected from:
1-(4-bromooxazol-2-yl)-7-phenylheptan-1-one;
1-(4-chlorooxazol-2-yl)-7-phenylheptan-1-one;
1-(4-iodooxazol-2-yl)-7-phenylheptan-1-one;
1-(4-methyloxazol-2-yl)-7-phenylheptan-1-one;
1-(4-(methylthio)oxazol-2-yl)-7-phenylheptan-1-ol;
1-(4-(methylthio)oxazol-2-yl)-7-phenylheptan-1-one;
2-(7-phenylheptanoyl)oxazole-4-carbaldehyde;
1-(4-acetyloxazol-2-yl)-7-phenylheptan-1-one;
7-phenyl-1-(4-(2,2,2-trifluoroacetyl)oxazol-2-yl)heptan-1-one;
methyl 2-(7-phenylheptanoyl)oxazole-4-carboxylate;
7-phenyl-1-(4-(pyridin-2-yl)oxazol-2-yl)heptan-1-one;
7-phenyl-1-(4-(pyridin-3-yl)oxazol-2-yl)heptan-1-one;
7-phenyl-1-(4-(pyridin-4-yl)oxazol-2-yl)heptan-1-one;
7-phenyl-1-(4-phenyloxazol-2-yl)heptan-1-one;
2-(7-phenylheptanoyl)oxazole-4-carboxylic acid;
2-(7-phenylheptanoyl)oxazole-4-carboxamide;
N-methyl-2-(7-phenylheptanoyl)oxazole-4-carboxamide;
N,N-dimethyl-2-(7-phenylheptanoyl)oxazole-4-carboxamide;
2-(7-phenylheptanoyl)oxazole-4-carbonitrile;
7-phenyl-1-(4-(trifluoromethyl)oxazol-2-yl)heptan-1-one; or
1-(4-methoxyoxazol-2-yl)-7-phenylheptan-1-one;
or a pharmaceutically acceptable salt, solvate, or hemiketal thereof.

11. A composition comprising a compound of claim 1 and a pharmaceutically acceptable diluent or carrier.

12. The composition of claim 11, further comprising an analgesic, wherein the analgesic is an opioid or a non-steroidal anti-inflammatory drug.

13. The composition of claim 11, further comprising an active ingredient selected from the group consisting of aspirin, acetaminophen, opioids, ibuprofen, naproxen, COX-2 inhibitors, gabapentin, pregabalin, and tramadol.

14. A method of treating a subject suffering from or diagnosed with a disease, disorder, or medical condition mediated by FAAH activity, comprising administering to the subject in need of such treatment an effective amount of at least one compound of formula I, a pharmaceutically acceptable salt thereof, a pharmaceutically acceptable prodrug thereof, or a pharmaceutically active metabolite thereof: 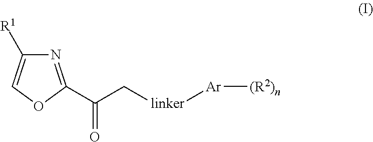
wherein
R¹ is —Y—R^{x};
Y is —CH₂—, —O—, —CH₂O—, —OCH₂—, —C(═O)—, —CO₂—, —OC(═O)—, —S—, —S(O)—, —S(O)₂—, —N(R^{a})—, or a direct bond;
R^{x} is H, halo, (C₁-C₂₀)alkyl, (C₁-C₈)cycloalkyl, trifluoromethyl, aryl, heteroaryl, —CN, —NO₂, or —NR^{a}R^{b};
linker is a (C₁-C₂₀)alkyl chain wherein one to five carbons of the chain are optionally be replaced with O or S, or linker is a direct bond;
Ar is (C₆-C₁₄)aryl;
each R² is independently H, —X—R³, or —X-Ph-X—R³;
n is 1-4;
each X is independently —CH₂—, —O—, —CH₂O—, —OCH₂—, —C(═O)—, —CO₂—, —OC(═O)—, —S—, —S(O)—, —S(O)₂—, —N(R^{a})—, or a direct bond;
each R³ is independently H, (C₁-C₈)alkyl, (C₃-C₈)cycloalkyl, aryl, heteroaryl, —CF₃, —CN, —C(O)(C₁-C₈)alkyl optionally substituted with one, two, or three fluoro substituents, —CO₂(C₁-C₈)alkyl, —CO₂H, —C(O)NR^{a}R^{b}, —OH, —O(C₁-C₈)alkyl, -halo, —NO₂, —NR^{a}R^{b}, —N(R^{a})C(O)R^{b}, —N(R^{a})SO₂R^{b}, —SO₂NR^{a}R^{b}, —S(O)₀₋₂R^{a}, or —CH₂NR^{c}R^{d} wherein R^{c} and R^{d} are each independently H or (C₁-C₈)alkyl, or R^{c} and R^{d} taken together with the nitrogen to which they are attached form a monocyclic saturated heterocyclic group;
each R^{a} and R^{b} are each independently H, (C₁-C₈)alkyl, (C₃-C₈)cycloalkyl, aryl(C₁-C₈)alkyl, or a nitrogen protecting group; and
any alkyl, cycloalkyl, aryl or heteroaryl of R^{x} is optionally substituted with one, two, or three R² groups.

15. The method of claim 14 wherein the compound of formula I is:
1-(4-bromooxazol-2-yl)-7-phenylheptan-1-one;
1-(4-chlorooxazol-2-yl)-7-phenylheptan-1-one;
1-(4-iodooxazol-2-yl)-7-phenylheptan-1-one;
1-(4-methyloxazol-2-yl)-7-phenylheptan-1-one;
1-(4-(methylthio)oxazole-2-yl)-7-phenylheptan-1-ol;
1-(4-(methylthio)oxazole-2-yl)-7-phenylheptan-1-one;
2-(7-phenylheptanoyl)oxazole-4-carbaldehyde;
1-(4-acetyloxazol-2-yl)-7-phenylheptan-1-one;
7-phenyl-1-(4-(2,2,2-trifluoroacetyl)oxazole-2-yl)heptan-1-one;
methyl 2-(7-phenylheptanoyl)oxazole-4-carboxylate;
7-phenyl-1-(4-(oxazole-2-yl)oxazole-2-yl)heptan-1-one;
7-phenyl-1-(4-(oxazole-3-yl)oxazole-2-yl)heptan-1-one;
7-phenyl-1-(4-(oxazole-4-yl)oxazole-2-yl)heptan-1-one;
7-phenyl-1-(4-phenyloxazol-2-yl)heptan-1-one;
2-(7-phenylheptanoyl)oxazole-4-carboxylic acid;
2-(7-phenylheptanoyl)oxazole-4-carboxamide;
N-methyl-2-(7-phenylheptanoyl)oxazole-4-carboxamide;
N,N-dimethyl-2-(7-phenylheptanoyl)oxazole-4-carboxamide;
2-(7-phenylheptanoyl)oxazole-4-carbonitrile;
7-phenyl-1-(4-(trifluoromethyl)oxazole-2-yl)heptan-1-one; or
1-(4-methoxyoxazol-2-yl)-7-phenylheptan-1-one;
or a pharmaceutically acceptable salt, solvate, or hemiketal thereof.

16. A method according to claim 14 or 15 wherein the disease, disorder, or medical condition comprises anxiety, depression, pain, sleep disorders, eating disorders, inflammation, movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, neuroinflammation, or a combination thereof.

17. The method of claim 16, wherein the disease, disorder, or medical condition is selected from the group consisting of anxiety, pain, inflammation, sleep disorders, eating disorders, and movement disorders.

18. A method of inhibiting fatty acid amide hydrolase activity comprising contacting the fatty acid amide hydrolase with an effective amount of a compound of claim 1.

19. The method of claim 18 wherein the contacting is in vivo.
